SEQUENCE LISTING (6 sequences)

SEQ ID NO: 1 - DNA, 23 bp, Artificial
  AMT31 transposon-specific primer
tgcagcaacgccaggtccacact

SEQ ID NO: 2 - DNA, 30 bp, Artificial
  AMT38 degenerate primer
  misc_feature (23)..(26): n is a, c, g, or t
gtaatacgactcactatagggcnnnncatg

SEQ ID NO: 3 - DNA, 24 bp, Artificial
  AMT32 nested primer
ctcttgctcttccgcttcttctcc

SEQ ID NO: 4 - DNA, 20 bp, Artificial
  AMT39 T7 primer
taatacgactcactataggg

SEQ ID NO: 5 - DNA, 19 bp, Artificial
  AMT 152 primer
ttgctcttccgcttcttct

SEQ ID NO: 6 - DNA, 19 bp, Artificial
  SP-6 primer
tatttaggtgacactatag

(19) United States
(12) Patent Application Publication
(10) Pub. No.: US 2007/0134708 A1
(43) Pub. Date: Jun. 14, 2007

(54) Diagnostic targets against Johne's disease

(76) Inventors: Adel Mohamed Talaat, Madison, WI (US); Chia-Wei Wu, Madison, WI (US)

Correspondence Address: BRINKS HOFER GILSON & LIONE, P.O. BOX 10395, CHICAGO, IL, 60610, US

(21) Appl. No.: 11/635,933

(22) Filed: Dec. 8, 2006

Related U.S. Application Data
(60) Provisional application No. 60/748,852, filed on Dec. 9, 2005.

(51) Int. Cl. C12Q 1/68; C07H 21/04

(52) U.S. Cl. 435/6; 536/23.7

(57) ABSTRACT

A composition and method for detecting Mycobacterium infection are disclosed. The gcpE, pstA, kdpC, papA2, impA, umaA1, fabG2₁₃2, aceAB, mbtH2, lpqP, map0834c, cspB, lipN, and map 1634 genes of M. paratuberculosis are novel virulence determinants for Johne's disease. Eighteen M. paratuberculosis-specific genomic islands were identified. Twenty-four M. avium-specific genomic islands were identified. Inversion of three large genomic fragments (INV) in M. paratuberculosis was also identified. These genomic identifiers represent novel virulence determinants that can be used as diagnostics targets for mycobacterial infection, and could provide suitable targets for vaccine and drug developments against Johne's disease.

### CROSS-REFERENCE TO RELATED APPLICATIONS

This invention claims priority to U.S. Provisional Patent Application Ser. No. 60/748,852 filed Dec. 9, 2005.

### GOVERNMENT INTERESTS

This invention was made with United States government support awarded by the following agency: USDA/CSREES grants 2004-35204-14209, 2004-35605-14243, 04-CRHF-0-6055. The United States may have certain rights in this invention.

### FIELD OF THE INVENTION

This invention relates to nucleic acid sequences from Mycobacterium avium subspecies paratuberculosis (hereinafter referred to as Mycobacterium paratuberculosis or M. paratuberculosis), the products encoded by those sequences, compositions containing those sequences and products, assays, and methods of diagnosis using those sequences and products.

### BACKGROUND OF THE INVENTION

Mycobacterium paratuberculosis causes Johne's disease (paratuberculosis) in dairy cattle. The disease is characterized by chronic diarrhea, weight loss, and malnutrition, resulting in estimated losses of $220 million per year in the USA alone. World-wide, the prevalence of the disease can range from as low as 3-4% of the examined herds in regions with low incidence (such as England), to high levels of 50% of the herds in some areas within the USA (Wisconsin and Alabama). Cows infected with Johne's disease are known to secrete Mycobacterium paratuberculosis in their milk. In humans, M. paratuberculosis bacilli have been found in tissues examined from Crohn's disease patients indicating possible zoonotic transmission from infected dairy products to humans.

Unfortunately, the virulence mechanisms controlling M. paratuberculosis persistence inside the host are poorly understood, and the key steps for establishing the presence of paratuberculosis are elusive. Mechanisms responsible for invasion and persistence of M. paratuberculosis inside the intestine remain undefined on a molecular level (Valentin-Weigand and Goethe, 1999, Microbes & Infection 1: 1121-1127). Both live and dead bacilli are observed in sub-epithelial macrophages after uptake. Once inside the macrophages, M. paratuberculosis survive and proliferate inside the phagosomes using unknown mechanisms.

M. paratuberculosis is closely related to Mycobacterium avium subspecies avium (hereinafter referred to as Mycobacterium avium or M. avium), which is a persistent health problem for immunocompromised humans, particularly HIV-positive individuals. Limited tools are available to researchers to definitively identify M. paratuberculosis and to distinguish it from M. avium. Existing methods are subject to high cross-reactivity, poor sensitivity, specificity, and predictive value. This dearth of knowledge translates into a lack of suitable vaccines for prevention and treatment of Johne's disease in animals, and of Crohn's disease in humans.

The current challenge in screening M. paratuberculosis is to identify those targets that are essential for survival of the bacilli during infection. Recently, random transposon mutagenesis-based protocols were employed for functional analysis of a large number of genes in M. paratuberculosis (Harris et al., 1999, FEMS Microbiology Letters 175: 21-26; Cavaignac et al., 2000, Archives of Microbiology 173: 229-231). When M. paratuberculosis was used as a target for mutagenesis, the libraries were screened to identify auxotrophs or genes responsible for survival under in vitro conditions. In these reports, six auxotrophs and two genes responsible for cell wall biosynthesis were identified (Harris et al., 1999; Cavaignac et al., 2000). So far, none of these libraries have been screened for virulence determinants.

Many clinical methods for detecting and identifying Mycobacterium species in samples require analysis of the bacterium's physical characteristics (e.g., acid-fast staining and microscopic detection of bacilli), physiological characteristics (e.g., growth on defined media) or biochemical characteristics (e.g., membrane lipid composition). These methods require relatively high concentrations of bacteria in the sample to be detected, may be subjective depending on the clinical technician's experience and expertise, and are time-consuming. Because Mycobacterium species are often difficult to grow in vitro and may take weeks to reach a useful density in culture, these methods can also result in delayed patient treatment and costs associated with isolating an infected individual until the diagnosis is completed.

More recently, assays that detect the presence of nucleic acid derived from bacteria in the sample have been preferred because of the sensitivity and relative speed of the assays. In particular, assays that use in vitro nucleic acid amplification of nucleic acids present in a clinical sample can provide increased sensitivity and specificity of detection. Such assays, however, can be limited to detecting one or a few Mycobacterium species depending on the sequences amplified and/or detected.

The genome sequences of both M. avium (Institute for Genomic Research, through the website at http://www.tigr.org) and of M. paratuberculosis (GenBank accession No. AE016958) are currently available. It would be useful to analyze these genomes to provide a higher resolution analysis of M. avium subspecies genomes. A better understanding of the virulence mechanisms and pathogenesis of M. paratuberculosis is required to develop more effective vaccine and chemotherapies directed against M. paratuberculosis. In view of the problems with bacterial specificity, the present inventors have focused their attention on identification of putative virulence factors that may contribute to the pathogenicity of M. paratuberculosis. This information could be used to design vaccines against pathogenic subspecies of M. avium. Such vaccines can be used for prevention and treatment of Johne's disease in animals or Crohn's disease in humans.

### SUMMARY OF THE INVENTION

This invention provides an isolated MAP genomic island from M. paratuberculosis. The isolated MAP genomic island may include a label. The MAP genomic island may be any one of MAP-1, MAP-2, MAP-3, MAP-4, MAP-5, MAP-6, MAP-7, MAP-8, MAP-9, MAP-10, MAP-11, MAP-12, MAP-13, MAP-14, MAP-15, MAP-16, MAP-17, or MAP-18, or homologs thereof.

This invention provides an isolated MAV genomic island from M. avium. The isolated MAV genomic island may include a label. The MAV genomic island may be any one of MAV-1, MAV-2, MAV-3, MAV-4, MAV-5, MAV-6, MAV-7, MAV-8, MAV-9, MAV-10, MAV-11, MAV-12, MAV-13, MAV-14, MAV-15, MAV-16, MAV-17, MAV-18, MAV-19, MAV-20, MAV-21, MAV-22, MAV-23, or MAV-24, or homologs thereof.

This invention provides a nucleic acid probe sequence comprising a nucleic acid sequence having at least 70% homology with any contiguous nucleotide sequence of at least 20 nucleotides that are substantially identical to the target sequence comprising at least one of: a) gcpE, pstA, kdpC, papA2, impA, umaA1, fabG2_{—}2, aceAB, mbtH2, lpqP, map0834c, cspB, lipN, or map1634 genes of M. paratuberculosis; b) MAP-1, MAP-2, MAP-3, MAP-4, MAP-5, MAP-6, MAP-7, MAP-8, MAP-9, MAP-10, MAP-11, MAP-12, MAP-13, MAP-14, MAP-15, MAP-16, MAP-17, or MAP-18; c) MAV-1, MAV-2, MAV-3, MAV-4, MAV-5, MAV-6, MAV-7, MAV-8, MAV-9, MAV-10, MAV-11, MAV-12, MAV-13, MAV-14, MAV-15, MAV-16, MAV-17, MAV-18, MAV-19, MAV-20, MAV-21, MAV-22, MAV-23, or MAV-24; or d) a junction sequence between an inverted genomic fragment INV and a flanking genomic island; or homologs thereof. The nucleic acid probe sequence may include a label.

This invention provides a method for detecting the presence or absence of a mycobacterial strain or phenotype in a test sample, which includes contacting a probe with a test sample. The probe includes a nucleic acid sequence having at least 70% homology with any contiguous nucleotide sequence of at least 20 nucleotides that are substantially identical to the target sequence comprising at least one of: (i) gcpE, pstA, kdpC, papA2, impA, umaA1, fabG2_{—}2, aceAB, mbtH2, lpqP, map0834c, cspB, lipN, or map 1634 genes of M. paratuberculosis; (ii) MAP-1, MAP-2, MAP-3, MAP-4, MAP-5, MAP-6, MAP-7, MAP-8, MAP-9, MAP-10, MAP-11, MAP-12, MAP-13, MAP-14, MAP-15, MAP-16, MAP-17, or MAP-18; (iii) MAV-1, MAV-2, MAV-3, MAV-4, MAV-5, MAV-6, MAV-7, MAV-8, MAV-9, MAV-10, MAV-11, MAV-12, MAV-13, MAV-14, MAV-15, MAV-16, MAV-17, MAV-18, MAV-19, MAV-20, MAV-21, MAV-22, MAV-23, or MAV-24; a junction sequence between an inverted genomic fragment INV and a flanking genomic island; or homologs thereof. The probe may be combined with a label. The method also includes analyzing for the presence, if any, of hybridized probe in the test sample, thereby detecting the presence or absence of a mycobacterial strain or phenotype in the test sample. The mycobacterial strain may be M. paratuberculosis. The mycobacterial strain may be M. avium. The mycobacterial strain may cause Johne's disease in animals or Crohn's disease in humans. The phenotype may be pathogenicity or drug resistance. The sample may comprise tissue, collection of cells, cell lysate, body fluid, excretum, in vitro culture, purified polynucleotide, isolated polynucleotide, food sample, medical sample, agro-livestock sample, or environmental sample.

For practicing the method, the target nucleic acid sequence may be a junction sequence between an inverted genomic fragment INV-1 and one of the two flanking genomic islands MAV-4 or MAV-19, or homologs thereof. The target nucleic acid sequence may be a junction sequence between an inverted genomic fragment INV-2 and one of the two flanking genomic islands MAV-21 and MAV-24, or homologs thereof. The target nucleic acid sequence may be a junction sequence between an inverted genomic fragment INV-3 and one of the two flanking genomic islands MAV-1 and MAV-2, or homologs thereof.

### BRIEF DESCRIPTION OF THE DRAWINGS

FIG. 1 is a schematic representation of the transposon Tn5367 from strain ATCC19698 used for insertion mutagenesis of M. paratuberculosis.

FIG. 2 depicts a genomic map showing the distribution of 1,128 transposon-insertion sites on the chromosome of M. paratuberculosis.

FIG. 3 depicts charts showing colonization levels of variable M. paratuberculosis strains to different mice organs.

FIG. 4 depicts charts showing intestinal colonization levels of variable M. paratuberculosis strains to different mice organs.

FIG. 5 depicts a chart showing the histopathology of mice infected with M. paratuberculosis strains.

FIG. 6 is a genomic map showing the identification of genomic islands in the M. avium genome (A), and a map showing the strategy used for design of PCR primers to confirm the genomic island deletions (B).

FIG. 7 is a genomic map showing the synteny of M. avium and M. paratuberculosis genomes.

### DETAILED DESCRIPTION OF THE INVENTION

The present invention provides genomic identifiers for mycobacterial species. These can be used as target nucleic acid sequences for diagnosis of mycobacterial infection. The diagnostic targets can be used for identification the presence of Johne's disease in a sample.

1. General Overview

The practice of the present invention employs, unless otherwise indicated, conventional techniques of molecular biology (including recombinant techniques), microbiology, cell biology, biochemistry, immunology, protein kinetics, and mass spectroscopy, which are within the skill of art. Such techniques are explained fully in the literature, such as Sambrook et al., 2000, Molecular Cloning: A Laboratory Manual, third edition, Cold Spring Harbor Laboratory Press; Current Protocols in Molecular Biology Volumes 1-3, John Wiley & Sons, Inc.; Kriegler, 1990, Gene Transfer and Expression: A Laboratory Manual, Stockton Press, New York; Dieffenbach et al., 1995, PCR Primer: A Laboratory Manual, Cold Spring Harbor Laboratory Press, each of which is incorporated herein by reference in its entirety. Procedures employing commercially available assay kits and reagents typically are used according to manufacturer-defined protocols unless otherwise noted.

Generally, the nomenclature and the laboratory procedures in recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications.

2. Definitions

The phrase “nucleic acid” or “polynucleotide sequence” refers to a single or double-stranded polymer of deoxyribonucleotide or ribonucleotide bases read from the 5′ to the 3′ end. Nucleic acids may also include modified nucleotides that permit correct read-through by a polymerase and do not alter expression of a polypeptide encoded by that nucleic acid.

The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be further understood that the sequence includes the degenerate codons of the native sequence or sequences which may be introduced to provide codon preference in a specific host cell.

A “coding sequence” or “coding region” refers to a nucleic acid molecule having sequence information necessary to produce a gene product, when the sequence is expressed.

“Homology” refers to the resemblance or similarity between two nucleotide or amino acid sequences. As applied to a gene, “homolog” may refer to a gene similar in structure and/or evolutionary origin to a gene in another organism or another species. As applied to nucleic acid molecules, the term “homolog” means that two nucleic acid sequences, when optimally aligned (see below), share at least 80 percent sequence homology, preferably at least 90 percent sequence homology, more preferably at least 95, 96, 97, 98 or 99 percent sequence homology. “Percentage nucleotide (or nucleic acid) homology” or “percentage nucleotide (or nucleic acid) sequence homology” refers to a comparison of the nucleotides of two nucleic acid molecules which, when optimally aligned, have approximately the designated percentage of the same nucleotides or nucleotides that are not identical but differ by redundant nucleotide substitutions (the nucleotide substitution does not change the amino acid encoded by the particular codon). For example, “95% nucleotide homology” refers to a comparison of the nucleotides of two nucleic acid molecules which, when optimally aligned, have 95% nucleotide homology.

A “genomic sequence” or “genome” refers to the complete DNA sequence of an organism. The genomic sequences of both M. avium and of M. paratuberculosis are known and are currently available. The genomic sequence of M. avium can be obtained from the Institute for Genomic Research, through the website http://www.tigr.org. The genomic sequence of M. paratuberculosis can be obtained from the GenBank, under accession number AE016958.

A “genomic island” (GI) refers to a nucleic acid region (and its homologs), that includes three or more consecutive open reading frames (ORFs), regardless of the size. A “MAP” genomic island means any genomic island (and its homologs) that is present in the M. paratuberculosis genome, but is not present in the M. avium genome. A “MAV” genomic island means any genomic island (and its homologs) that is present in the M. avium-genome, but is not present in the M. paratuberculosis genome.

A “junction” between two nucleic acid regions refers to a point that joins two nucleic acid regions. A “junction sequence” refers to a nucleic acid sequence that can be used for identification of the junction point. For example, a “junction sequence”, or a “junction region” of an inverted region (INV) and a corresponding flanking sequence refers to a nucleic acid segment that crosses the point that joins the inverted region with the flanking sequence. Such a nucleic acid segment is specific to the corresponding junction region (junction sequence), and can be used as its identifier.

The term “nucleic acid construct” or “DNA construct” is sometimes used to refer to a coding sequence or sequences operably linked to appropriate regulatory sequences so as to enable expression of the coding sequence, and inserted into a expression cassette for transforming a cell. This term may be used interchangeably with the term “transforming DNA” or “transgene”. Such a nucleic acid construct may contain a coding sequence for a gene product of interest, along with a selectable marker gene and/or a reporter gene.

A “label” is a composition detectable by spectroscopic, photochemical, biochemical, immunochemical, or chemical means. Useful labels include ³²P, fluorescent dyes, electron-dense reagents, enzymes (e.g., as commonly used in an ELISA), biotin, digoxigenin, or proteins for which antisera or monoclonal antibodies are available. For example, labels are preferably covalently bound to a genomic island, directly or through the use of a linker.

A “nucleic acid probe sequence” or “probe” is defined as a nucleic acid capable of binding to a target nucleic acid of complementary sequence through one or more types of chemical bonds, usually through complementary base pairing, usually through hydrogen bond formation. A probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. Probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “recombinant” when used with reference, e.g., to a cell, or nucleic acid, protein, expression cassette, or vector, indicates that the cell, nucleic acid, protein, expression cassette, or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, underexpressed, or not expressed at all.

“Antibodies” refers to polyclonal and monoclonal antibodies, chimeric, and single chain antibodies, as well as Fab fragments, including the products of a Fab or other immunoglobulin expression library. With respect to antibodies, the term, “immunologically specific” refers to antibodies that bind to one or more epitopes of a protein of interest, but which do not substantially recognize and bind other molecules in a sample containing a mixed population of antigenic biological molecules. The present invention provides antibodies immunologically specific for part or all of the polypeptides of the present invention, e.g., those polypeptides encoded by the genes gcpE, pstA, kdpC, papA2, impA, umaA1, fabG2_{—}2, aceAB, mbtH2, lpqP, map0834c, cspB, lipN, and map1634 of Mycobacterium paratuberculosis.

An “expression cassette” refers to a nucleic acid construct, which when introduced into a host cell, results in transcription and/or translation of a RNA or polypeptide, respectively. Expression cassettes can be derived from a variety of sources depending on the host cell to be used for expression. An expression cassette can contain components derived from a viral, bacterial, insect, plant, or mammalian source. In the case of both expression of transgenes and inhibition of endogenous genes (e.g., by antisense, or sense suppression) the inserted polynucleotide sequence need not be identical and can be “substantially identical” to a sequence of the gene from which it was derived.

The term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include other forms of expression vectors, such as viral vectors (e.g., replication defective retroviruses, adenoviruses and adeno-associated viruses), which serve equivalent functions.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated nucleic acid of the present invention is separated from open reading frames that flank the desired gene and encode proteins other than the desired protein. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

In the case where the inserted polynucleotide sequence is transcribed and translated to produce a functional polypeptide, because of codon degeneracy a number of polynucleotide sequences will encode the same polypeptide. These variants are specifically covered by the term “polynucleotide sequence from” a particular gene. In addition, the term specifically includes sequences (e.g., full length sequences) substantially identical (determined as described below) with a gene sequence encoding a protein of the present invention and that encode proteins or functional fragments that retain the function of a protein of the present invention, e.g., a disease causing agent of M. paratuberculosis.

In the case of polynucleotides used to identify an endogenous gene, the probe sequence need not be perfectly identical to a sequence of the target endogenous gene. The probe polynucleotide sequence will typically be at least substantially identical (as determined below) to the target endogenous sequence.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

Optimal alignment of sequences for comparison may be conducted by methods commonly known in the art, e.g., the local homology algorithm (Smith and Waterman, 1981, Adv. Appl. Math. 2: 482-489), by the search for similarity method (Pearson and Lipman 1988, Proc. Natl. Acad. Sci. USA 85: 2444-2448), by computerized implementations of these algorithms (GAP, BESTFIT, BLAST, FASTA, and TFASTA in the Wisconsin Genetics Software Package, Genetics Computer Group (GCG), Madison, Wis.), or by inspection.

Protein and nucleic acid sequence identities are evaluated using the Basic Local Alignment Search Tool (“BLAST”) which is well known in the art (Karlin and Altschul, 1990, Proc. Natl. Acad. Sci. USA 87: 2267-2268; Altschul et al., 1997, Nucl. Acids Res. 25: 3389-3402). The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990), the disclosure of which is incorporated by reference in its entirety. The BLAST programs can be used with the default parameters or with modified parameters provided by the user.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 25% sequence identity. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like.

“Substantial identity” of amino acid sequences for purposes of this invention normally means polypeptide sequence identity of at least 40%. Preferred percent identity of polypeptides can be any integer from 40% to 100%. More preferred embodiments include at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 98.7%, or 99%.

Polypeptides that are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

3. Identification of Nucleic Acids of the Present Invention

The present invention relates to a method for detecting the presence or amount of a target polynucleotide (nucleic acid sequence) from Mycobacterium paratuberculosis in a sample. The target polynucleotide is a virulence determinant. The invention is also directed to a method of detecting the presence of a disease state in a mammal, by detecting the presence or amount of a target polynucleotide, wherein the presence or amount of the target polynucleotide identifies the disease state. Thus, the invention relates to diagnostic compositions and methods for detecting Johne's disease. The sample containing the target polynucleotide may be tissue, collection of cells, cell lysate, body fluid, excretum, in vitro culture, purified polynucleotide, isolated polynucleotide, food sample, medical sample, agro-livestock sample, or environmental sample.

The invention described here utilizes large-scale identification of disrupted genes and the use of bioinformatics to select mutants that could be characterized in animals. Employing such an approach, novel virulence determinants were identified, based on mutants that were investigated in mice. These virulence determinants can be used for designing vaccines. Compared to similar protocols established for identifying virulence genes such as signature-tagged mutagenesis (Ghadiali et al., 2003, Nucleic Acids Res. 31: 147-151), the approach employed here is simpler and uses a smaller number of animals.

The target nucleic acid sequences of the present invention include the gcpE, pstA, kdpC, papA2, impA, umaA1, fabG2_{—}2, aceAB, mbtH2, lpqP, map0834c, cspB, lipN, and map 1634 genes of M. paratuberculosis, their homologs, and the corresponding gene products. Presence of these genes, their homologs, and/or their products in a sample is indicative of a M. paratuberculosis infection.

The start and end coordinates of the M. paratuberculosis polynucleotides of this invention (e.g., genes, genomic islands, inverted regions, junction sequences) are based on the genomic sequence of M. paratuberculosis strain K10 (Li et al., 2005, Proc. Natl. Acad. Sci. USA 102: 12344-12349; GenBank No. AE016958). The start and end coordinates of the M. avium polynucleotides of this invention (e.g., genes, genomic islands, inverted regions, junction sequences) are based on the genomic sequence of M. avium strain 104, as obtained from The Institute for Genomic Research through the website at http://www.tigr.org.

The size of gcpE is 1167 base pairs (bp), and it is located at positions 3272755 through 3273921 of the M. paratuberculosis genomic sequence.

The size of pstA is 12084 base pairs (bp), and it is located at positions 1309241 through 1321324 of the M. paratuberculosis genomic sequence.

The size of kdpC is 876 base pairs (bp), and it is located at positions 1038471 through 1039346 of the M. paratuberculosis genomic sequence.

The size of papA2 is 1518 base pairs (bp), and it is located at positions 1854059 through 1855576 of the M. paratuberculosis genomic sequence.

The size of umaA1 is 861 base pairs (bp), and it is located at positions 4423752 through 4424612 of the M. paratuberculosis genomic sequence.

The size of fabG2_{—}2 is 750 base pairs (bp), and it is located at positions 2704522 through 2705271 of the M. paratuberculosis genomic sequence.

The size of aceAB is 2288 base pairs (bp), and it is located at positions 1795784 through 1798072 of the M. paratuberculosis genomic sequence.

The size of mbtH2 is 233 base pairs (bp), and it is located at positions 2063983 through 2064216 of the M. paratuberculosis genomic sequence.

The size of IpqP is 971 base pairs (bp), and it is located at positions 4755529 through 4756500 of the M. paratuberculosis genomic sequence.

The size of map0834c is 701 base pairs (bp), and it is located at positions 851908 through 852609 of the M. paratuberculosis genomic sequence.

The size of map1634 is 917 base pairs (bp), and it is located at positions 1789023 through 1789940 of the M. paratuberculosis genomic sequence.

In another aspect, the target polynucleotides of the present invention that are virulence determinants include genomic islands. These GIs are strain-specific. The inventors have identified 18 M. paratuberculosis-specific genomic islands (MAPs), that are absent from the M. avium genome (Table 8).

The size of MAP-1 is 19,343 base pairs (bp). MAP-1 includes 17 ORFs. MAP-1 is located at positions 99947 through 119289 of the M. paratuberculosis genomic sequence.

The size of MAP-2 is 3,858 base pairs (bp). MAP-2 includes 3 ORFs. MAP-2 is located at positions 299412 through 303269 of the M. paratuberculosis genomic sequence.

The size of MAP-3 is 2,915 base pairs (bp). MAP-3 includes 3 ORFs. MAP-3 is located at positions 410091 through 413005 of the M. paratuberculosis genomic sequence.

The size of MAP-4 is 16,681 base pairs (bp). MAP-4 includes 17 ORFs. MAP-4 is located at positions 872772 through 889452 of the M. paratuberculosis genomic sequence.

The size of MAP-5 is 14,191 base pairs (bp). MAP-5 includes 17 ORFs. MAP-5 is located at positions 989744 through 1003934 of the M. paratuberculosis genomic sequence.

The size of MAP-6 is 8,971 base pairs (bp). MAP-6 includes 6 ORFs. MAP-6 is located at positions 1291689 through 1300659 of the M. paratuberculosis genomic sequence.

The size of MAP-7 is 6,914 base pairs (bp). MAP-7 includes 6 ORFs. MAP-7 is located at positions 1441777 through 1448690 of the M. paratuberculosis genomic sequence.

The size of MAP-8 is 7,915 base pairs (bp). MAP-8 includes 8 ORFs. MAP-8 is located at positions 1785511 through 1793425 of the M. paratuberculosis genomic sequence.

The size of MAP-9 is 11,202 base pairs (bp). MAP-9 includes 10 ORFs. MAP-9 is located at positions 1877255 through 1888456 of the M. paratuberculosis genomic sequence.

The size of MAP-10 is 2,993 base pairs (bp). MAP-10 includes 3 ORFs. MAP-10 is located at positions 1891000 through 1893992 of the M. paratuberculosis genomic sequence.

The size of MAP-11 is 2,989 base pairs (bp). MAP-11 includes 4 ORFs. MAP-11 is located at positions 2233123 through 2236111 of the M. paratuberculosis genomic sequence.

The size of MAP-12 is 11,977 base pairs (bp). MAP-12 includes 11 ORFs. MAP-12 is located at positions 2378957 through 2390933 of the M. paratuberculosis genomic sequence.

The size of MAP-13 is 19,977 base pairs (bp). MAP-13 includes 19 ORFs. MAP-13 is located at positions 2421552 through 2441528 of the M. paratuberculosis genomic sequence.

The size of MAP-14 is 19,315 base pairs (bp). MAP-14 includes 19 ORFs. MAP-14 is located at positions 3081906 through 3101220 of the M. paratuberculosis genomic sequence.

The size of MAP-15 is 4,143 base pairs (bp). MAP-15 includes 3 ORFs. MAP-15 is located at positions 3297661 through 3301803 of the M. paratuberculosis genomic sequence.

The size of MAP-16 is 79,790 base pairs (bp). MAP-16 includes 56 ORFs. MAP-16 is located at positions 4140311 through 4220100 of the M. paratuberculosis genomic sequence.

The size of MAP-17 is 3,655 base pairs (bp). MAP-17 includes 5 ORFs. MAP-17 is located at positions 4735049 through 4738703 of the M. paratuberculosis genomic sequence.

The size of MAP-18 is 3,512 base pairs (bp). MAP-18 includes 3 ORFs. MAP-18 is located at positions 4800932 through 4804443 of the M. paratuberculosis genomic sequence.

The inventors have also identified 24 M. avium-specific genomic islands (MAVs), that are absent from the M. paratuberculosis genome (Table 9).

The size of MAV-1 is 39,833 base pairs (bp). MAV-1 includes 38 ORFs. MAV-1 is located at positions 254394 through 294226 of the M. avium genomic sequence.

The size of MAV-2 is 31,387 base pairs (bp). MAV-2 includes 32 ORFs. MAV-2 is located at positions 461414 through 492800 of the M. avium genomic sequence.

The size of MAV-3 is 9,693 base pairs (bp). MAV-3 includes 10 ORFs. MAV-3 is located at positions 666033 through 675725 of the M. avium genomic sequence.

The size of MAV-4 is 47,356 base pairs (bp). MAV-4 includes 53 ORFs. MAV-4 is located at positions 747095 through 794450 of the M. avium genomic sequence.

The size of MAV-5 is 17,905 base pairs (bp). MAV-5 includes 16 ORFs. MAV-5 is located at positions 1421722 through 1439626 of the M. avium genomic sequence.

The size of MAV-6 is 19,161 base pairs (bp). MAV-6 includes 23 ORFs. MAV-6 is located at positions 1444205 through 1463365 of the M. avium genomic sequence.

The size of MAV-7 is 196,411 base pairs (bp). MAV-7 includes 187 ORFs. MAV-7 is located at positions 1795281 through 1991691 of the M. avium genomic sequence.

The size of MAV-8 is 2,977 base pairs (bp). MAV-8 includes 3 ORFs. MAV-8 is located at positions 2097907 through 2100883 of the M. avium genomic sequence.

The size of MAV-9 is 20,844 base pairs (bp). MAV-9 includes 15 ORFs. MAV-9 is located at positions 2220320 through 2241163 of the M. avium genomic sequence.

The size of MAV-10 is 12,491 base pairs (bp). MAV-10 includes 12 ORFs. MAV-10 is located at positions 2259120 through 2271610 of the M. avium genomic sequence.

The size of MAV-11 is 3,593 base pairs (bp). MAV-11 includes 5 ORFs. MAV-11 is located at positions 2462693 through 2466285 of the M. avium genomic sequence.

The size of MAV-12 is 181,445 base pairs (bp). MAV-12 includes 168 ORFs. MAV-12 is located at positions 2549555 through 2730999 of the M. avium genomic sequence.

The size of MAV-13 is 5,525 base pairs (bp). MAV-13 includes 7 ORFs. MAV-13 is located at positions 2815625 through 2821149 of the M. avium genomic sequence.

The size of MAV-14 is 28,265 base pairs (bp). MAV-14 includes 26 ORFs. MAV-14 is located at positions 3008716 through 3036980 of the M. avium genomic sequence.

The size of MAV-15 is 4,731 base pairs (bp). MAV-15 includes 3 ORFs. MAV-15 is located at positions 3214820 through 3219550 of the M. avium genomic sequence.

The size of MAV-16 is 44,157 base pairs (bp). MAV-16 includes 6 ORFs. MAV-16 is located at positions 3340393 through 3384549 of the M. avium genomic sequence.

The size of MAV-17 is 21,219 base pairs (bp). MAV-17 includes 20 ORFs. MAV-17 is located at positions 3392586 through 3413804 of the M. avium genomic sequence.

The size of MAV-18 is 3,918 base pairs (bp). MAV-18 includes 4 ORFS. MAV-18 is located at positions 3523417 through 3527334 of the M. avium genomic sequence.

The size of MAV-19is5,169 base pairs (bp). MAV-19 includes 4 ORFs. MAV-19 is located at positions 3670518 through 3675686 of the M. avium genomic sequence.

The size of MAV-20 is 21,283 base pairs (bp). MAV-20 includes 15 ORFs. MAV-20 is located at positions 3917752 through 3939034 of the M. avium genomic sequence.

The size of MAV-21 is 6,895 base pairs (bp). MAV-21 includes 8 ORFs. MAV-21 is located at positions 4254594 through 4261488 of the M. avium genomic sequence.

The size of MAV-22 is 9,931 base pairs (bp). MAV-22 includes 9 ORFs. MAV-22 is located at positions 5122371 through 5132301 of the M. avium genomic sequence.

The size of MAV-23 is 95,547 base pairs (bp). MAV-23 includes 77 ORFs. MAV-23 is located at positions 5174641 through 5270187 of the M. avium genomic sequence.

The size of MAV-24 is 16,200 base pairs (bp). MAV-24 includes 18 ORFs. MAV-24 is located at positions 5378903 through 5395102 of the M. avium genomic sequence.

The GIs of the present invention (both MAPs and MAVs) can be used as target nucleic acid sequences for diagnostic purposes. Thus, the targets enable one skilled in the art to distinguish between the presence of M. paratuberculosis or M. avium in a sample. Should both Mycobacterium strains be present in a sample, one should be able to identify the presence of both classes of target polynucleotides in the sample.

It is possible to diagnose the presence of M. paratuberculosis or M. avium in a sample due to the inversion of three large genomic fragments in M. paratuberculosis in comparison to M. avium. It was unexpectedly discovered that, when the GIs associated with both genomes were aligned, three large genomic fragments in M. paratuberculosis were identified as inverted relative to the corresponding genomic fragments in M. avium. These inverted nucleic acid regions (INV) had the sizes of approximately 54.9 Kb, 863.8 Kb and 1,969.4 Kb (FIG. 8).

The largest inverted region (INV-1 of approximately 1969.4 Kb) is flanked by MAV-4 and MAV-19. INV-1 encompasses bases 1075033 through 3044433 of the M. paratuberculosis genomic sequence.

The second inverted region (INV-2, of approximately 863.8 Kb) is flanked by MAV-21 and MAV-24, near the origin of replication in both genomes. INV-2 encompasses bases 3885218 through 4748979 of the M. paratuberculosis genomic sequence.

The smallest inversion (INV-3, of approximately 54.9 Kb) is flanked by MAV-1 and MAV-2. INV-3 encompasses bases 320484 through 377132 of the M. paratuberculosis genomic sequence.

One skilled in the art will know to detect the junctions of the inverted regions and the corresponding flanking sequences, thereby identifying the strain, and diagnosing the presence or absence of Johne's disease. For example, one can design probes that are directed to the junctions (sequences) of the INV regions and the corresponding flanking MAV sequences. Because the junction sequences are strain-specific, one will be able to distinguish between the presence of M. avium or M. paratuberculosis in a sample.

The target polynucleotide may be DNA. In some variations, the target polynucleotide may be obtained from total cellular DNA, or in vitro amplified DNA.

The invention also relates to nucleic acids that selectively hybridize to the exemplified target polynucleotide sequences, including hybridizing to the exact complements of these sequences. Such nucleic acids are referred to as “nucleic acid probe sequences” or “probes”. The specificity of single stranded DNA to hybridize complementary fragments is determined by the “stringency” of the reaction conditions. Hybridization stringency increases as the propensity to form DNA duplexes decreases. In nucleic acid hybridization reactions, the stringency can be chosen to either favor specific hybridizations (high stringency), which can be used to identify, for example, full-length clones from a library. Less-specific hybridizations (low stringency) can be used to identify related, but not exact, DNA molecules (homologous, but not identical) or segments.

The nucleic acid probe sequence (probe) may be partially complementary to the target nucleic acid sequence. Alternatively, the nucleic acid probe sequence may be exactly complementary to the target nucleic acid sequence. The nucleic acid probe sequence may be greater than about 4 nucleic acid bases in length and/or less than about 48 nucleic acid bases in length. In a further variation, the nucleic acid probe sequence may also be about 20 nucleic acid bases in length.

The nucleic acid probe sequence or target polynucleotide may be immobilized on a solid substrate. Immobilization may be via a non-covalent interaction, such as between biotin and streptavidin. In a further variation, the nucleic acid probe sequence may be covalently linked to biotin.

Identification of target sequences of the present invention may be accomplished by a number of techniques. For instance, oligonucleotide probes based on the sequences disclosed here can be used to identify the desired gene in a cDNA or genomic DNA library from a desired bacterial strain. To construct genomic libraries, large segments of genomic DNA are generated by random fragmentation, e.g. using restriction endonucleases, and are ligated with vector DNA to form concatemers that can be packaged into the appropriate vector. The cDNA or genomic library can then be screened using a probe based upon the sequence of a cloned gene such as the polynucleotides disclosed here. Probes may be used to hybridize with genomic DNA or cDNA sequences to identify homologous genes in the same or different bacterial strains.

Alternatively, the nucleic acids of interest can be amplified from nucleic acid samples using amplification techniques. For instance, polymerase chain reaction (PCR) technology can be used to amplify the sequences of the genes directly from mRNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes.

Appropriate primers and probes for identifying the target sequences of the present invention from a sample are generated from comparisons of the sequences provided herein, according to standard PCR guides. For examples of primers used see the Examples section below.

Polynucleotides may also be synthesized by well-known techniques described in the technical literature. Double-stranded DNA fragments may then be obtained either by synthesizing the complementary strand and annealing the strands together under appropriate conditions, or by adding the complementary strand using DNA polymerase with an appropriate primer sequence.

Once a nucleic acid is isolated using the method described above, standard methods can be used to determine if the nucleic acid is a preferred nucleic acid of the present invention, e.g., by using structural and functional assays known in the art. For example, using standard methods, the skilled practitioner can compare the sequence of a putative nucleic acid sequence thought to encode a preferred protein of the present invention to a nucleic acid sequence encoding a preferred protein of the present invention to determine if the putative nucleic acid is a preferred polynucleotide of the present invention.

Gene amplification and/or expression can be measured in a sample directly, for example, by conventional Southern blotting, Northern blotting to quantitate the transcription of mRNA, dot blotting (DNA analysis), DNA microarrays, or in situ hybridization, using an appropriately labeled probe, based on the sequences provided herein. Various labels can be employed, most commonly fluorochromes and radioisotopes, particularly ³²P. However, other techniques can also be employed, such as using biotin-modified nucleotides for introduction into a polynucleotide. The biotin then serves as the site for binding to avidin or antibodies, which can be labeled with a variety of labels, such as radionuclides, fluorescers, enzymes, or the like. Alternatively, antibodies can be employed that can recognize specific duplexes, including DNA duplexes, RNA duplexes, DNA-RNA hybrid duplexes or DNA-protein duplexes. The antibodies in turn can be labeled and the assay can be carried out where the duplex is bound to a surface, so that upon the formation of duplex on the surface, the presence of antibody bound to the duplex can be detected.

Gene expression can also be measured by immunological methods, such as immunohistochemical staining. With immunohistochemical staining techniques, a sample is prepared, typically by dehydration and fixation, followed by reaction with labeled antibodies specific for the gene product coupled, where the labels are usually visually detectable, such as enzymatic labels, fluorescent labels, luminescent labels, and the like. Gene expression can also be measured using PCR techniques, or using DNA microarrays, commonly known as gene chips.

The present invention also provides for antibodies immunologically specific for all or part, e.g., an amino-terminal portion, of a polypeptide at least 70% identical to a sequence that is a virulence determinant.

The invention is also directed to kits for detecting a target polynucleotide. The kit may include one or more of a sample that includes a target polynucleotide, and one or more nucleic acid probe sequences at least partially complementary to a target nucleic acid sequence. The kit may include instructions for using the kit.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

It is to be understood that this invention is not limited to the particular methodology, protocols, patients, or reagents described, and as such may vary. It is also to be understood that the terminology used herein is for the purpose of describing particular embodiments only, and is not intended to limit the scope of the present invention, which is limited only by the claims. The following examples are offered to illustrate, but not to limit the claimed invention.

### Example 1

Animals

Groups of BALB/c mice (N=10-20) at 3 to 4 weeks of age were infected with M. paratuberculosis strains using intraperitoneal (IP) injection. Infected mice were sacrificed at 3, 6 and 12 weeks post-infection and their livers, spleens and intestines collected for both histological and bacteriological examinations. Tissue sections collected for histopathology were preserved in 10% neutralized buffer formalin (NBF) before embedding in paraffin, cut into 4-5 μm sections, stained with hematoxylin and eosin (HE) or acid fast staining (AFS). Tissue sections from infected animals were examined by two independent pathologists at 3, 6 and 12 weeks post infection. The severity of inflammatory responses was ranked using a score of 0 to 5 based on lesion size and number per field. Tissues with more than 3 fields containing multiple, large-sized lesions were given a score of 5 using the developed scale.

Bacterial Strains, Cultures and Vectors

Mycobacterium avium subsp. paratuberculosis strain ATCC 19698 (M. paratuberculosis) was used for constructing the mutant library. This strain was grown at 37° C. in Middlebrook 7H9 broth enriched with 10% albumin dextrose complex (ADC), 0.5% glycerol, 0.05% Tween 80 and 2 mg/ml of mycobactin J (Allied Monitor, Ind.).

The temperature-sensitive, conditionally replicating phasmid (phAE94) used to deliver the transposon Tn5367 was obtained from Bill Jacobs laboratory (Albert Einstein College of Medicine) and propagated in Mycobacterium smegmatis mc² 155 at 30° C. as described previously (Bardarov et al., 1997, Proc. Natl. Acad. Sci. USA 94: 10961-10966). The Tn5367 is an IS1096-derived insertion element containing a kanamycin resistance gene as a selectable marker.

After phage transduction, mutants were selected on Middlebrook 7H10 medium plates supplemented with 30 μg/ml of kanamycin. Escherichia coli DH5α cells used for cloning purposes were grown on Luria-Bertani (LB) agar or broth supplemented with 100 μg /ml ampicillin. The plasmid vector pGEM T-easy (Promega, Madison, Wis.) was used for TA cloning the PCR products before sequencing.

Construction of a Transposon Mutants Library

The phasmid phAE94 was used to deliver the Tn5367 to mycobacterial cells using a protocol established earlier for M. tuberculosis. For each transduction, 10 ml of M. paratuberculosis culture was grown to 2×10⁸ CFU/ml (OD₆₀₀ 0.6-0.8), centrifuged and resuspended in 2.5 ml of MP buffer (50 mM Tris-HCl [pH 7.6], 150 mM NaCl, 2 mM CaCl₂) and incubated with 10¹⁰ PFU of phAE94 at the non-permissive temperature (37° C.) for 2 h in a shaking incubator to inhibit a possible lytic or lysogenic cycle of the phage.

Adsorption stop buffer (20 mM sodium citrate and 0.2% Tween 80) was added to prevent further phage infections and this mixture was plated immediately on 7H10 agar supplemented with 30 μg/ml of kanamycin and incubated at 37° C. for 6 weeks. Kanamycin-resistant colonies (5,060) were inoculated into 2 ml of 7H9 broth supplemented with kanamycin in 96-well microtitre plates for additional analysis.

Construction of lipN mutant. The lipN gene was deleted from M. paratuberculosis K10 strain using a homologous recombination protocol based on phage transduction. The whole gene was deleted from M. paratuberculosis K10 and was tested in mice. This gene was selected for deletion because of its up-regulation when DNA microarrays were used to analyze in vivo (fecal samples) collected from infected cows with high levels of mycobacterial shedding.

Southern Blot Analysis

To examine the randomness of Tn5367 insertions in the M. paratuberculosis genome, 10 randomly selected mutants were analyzed by Southern blot using a standard protocol. Kanamycin-resistant M. paratuberculosis single colonies were grown separately in 10 ml of 7H9 broth for 10 days at 37° C. before genomic DNA extraction and digestion (2-3 μg) with BamH1 restriction enzyme. Digested DNA fragments from both mutant and wild-type strains were electrophoresed on a 1% agarose gel and transferred to a nylon membrane (Perkin Elmer, Calif.), using an alkaline transfer protocol as recommended by the manufacturer.

A 1.3-kb DNA fragment from the kanamycin resistance gene was radiolabeled with [α-³²P]-dCTP using a Random Prime Labeling Kit (Promega) in accordance with the manufacture's direction. The radio-labeled probe was hybridized to the nylon membrane at 65° C. overnight in a shaking water bath before washing, exposure to X-ray film, and development to visualize hybridization signals.

Sequencing of the Transposon Insertion Site

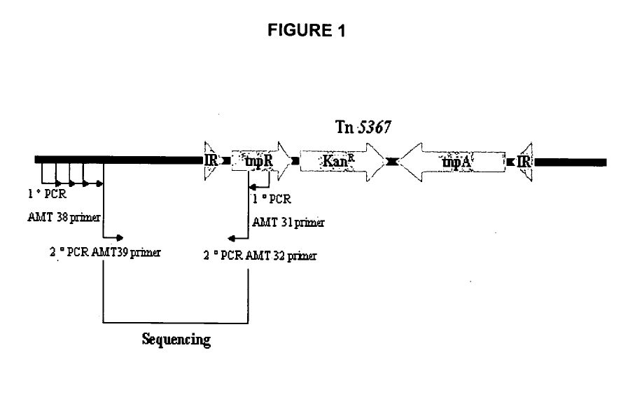

FIG. 1 shows a schematic representation of the transposon Tn5367 from strain ATCC19698 used for insertion mutagenesis of M. paratuberculosis. To determine the exact transposon insertion site within the M. paratuberculosis genome, a protocol for sequencing randomly primed PCR products was adopted from previous work on M. tuberculosis with slight modifications. For PCR amplification, the genomic DNA of each mutant was extracted from individual cultures by boiling for 10 min, centrifuged at 10,000 ×g for 1 min, and 10 μl of the supernatants were used in a standard PCR reaction. For the first round of PCR, a transposon-specific primer (AMT31: 5′-TGCAGCMCGCCAGGTCCACACT-3′) (SEQ ID NO:1) and the degenerate primer (AMT38: 5′-GTAATACGACTCACTATAGGGCNNNNCATG-3′) (SEQ ID NO:2) were used to amplify the chromosomal sequence flanking the transposon-insertion site.

PCR was carried out in a total volume of 25 μl in 10 mM Tris/HCl (pH 8.3), 50 mM KCl,2.0 mM MgCl₂, 0.01% (w/v) BSA, 0.2 mMdNTPs, 0.1 μM of primer AMT31, 1.0 μM of primer AMT38 and 0.75 U Taq polymerase (Promega). First-round amplification was performed with an initial denaturing step at 94° C. for 5 min, followed by 40 cycles of denaturing at 94° C. for 1 min, annealing at 50° C. for 30 s and extension at 72° C. for 90 s, with a final extension step at 72° C. for 7 min. Only 1 μl of the first round amplification was then used as a template for the second round PCR (nested PCR) using a nested primer (AMT32: 5′-CTCTTGCTCTTCCGCTTCTTCTCC-3′) (SEQ ID NO:3) derived from the Tn5367 and T7 primer (AMT 39: 5′-TMTACGACTCACTATAGGG-3′) (SEQ ID NO:4) present within the degenerative primer sequence. Reactions were carried out in a total volume of 50 μl in 10 mM Tris/HCl (pH 8.3), 50 mM KCl, 1.5 mM MgCl₂, 0.2 mM dNTPs, 0.5 μM primers, 5% (v/v) DMSO and 0.75 U Taq polymerase.

A final round of amplification was performed with a denaturing step at 95° C. for 5 min followed by 35 thermocycles (94° C. for 30 s, 57° C. for 30 s and 72° C. for 1 min) with a final extension step at 72° C. for 10 min. For almost ⅔ of the sequenced mutants, no cloning was attempted and AMT152 primer (5′-TTGCTCTTCCGCTTCTTCT-3′) (SEQ ID NO:5) present in Tn5367 was used to directly sequence gel-purified amplicons. The product of the second amplification was gel-purified (Wizard Gel-extraction kit, Promega, Madison, Wis.) and cloned into pGEM T-easy vector for plasmid mini-preparation followed by automatic sequencing. Inserts in pGEM T-easy vector was confirmed by EcoRI restriction digestion and the sequencing was carried out using SP-6 primer (5′-TATTTAGGTGACACTATAG-3′) (SEQ ID NO:6).

To identify the precise transposon-insertion site in the M. paratuberculosis genome, the transposon sequence was trimmed from the cloning vector sequences and a BLASTN search was used against the M. paratuberculosis K-10 complete genome sequence (GenBank accession no. AE016958). Sequences with at least 100 bp of alignment matching to the M. paratuberculosis genome were further analyzed while others without any transposon sequence were not analyzed to avoid using amplicons generated by non-specific primer binding and amplification.

Statistical Analysis

All bacterial counts from mouse organs were statistically analyzed using the Excel program (Microsoft, Seattle, Wash.). All counts are expressed as the mean±standard deviation (S.D.). Differences in counts between groups were analyzed with a Student's t-test for paired samples. Differences were considered to be significant if a probability value of p<0.05 was obtained when the CFU count of mutant strains were compared to that of the wild-type strain.

Generation of M. paratuberculosis Mutant Library

A genome-wide random-insertion mutant library was generated for the M. paratuberculosis ATCC 19698 using the temperature-sensitive mycobacteriophage phAE94 developed earlier for M. tuberculosis. A library consisting of 5,060 kanamycin-resistance colonies was obtained by the insertion of transposon Tn5367 in the bacterial genome (FIG. 1). One transduction reaction of 10⁹ mycobacterial cells with phAE94 yielded all of the kanamycin resistant colonies used throughout this study. None of the retrieved colonies displayed a variant colony morphology from that usually observed in members of the M. avium complex. A large-scale sequencing strategy was employed to identify disrupted genes.

Identification of the Transposition Sites in M. paratuberculosis Mutants

Among the library of 5,060 mutants, 1,150 were analyzed using a high-throughput sequencing analysis employing a randomly primed PCR protocol that was successful in characterizing an M. tuberculosis-transposon library. These sequences were used to search M. paratuberculosis K-10 complete genome using BLASTN algorithm to identify the insertion site in 20% of the library. Generally, unique insertion sites (N=970) were identified, and almost ⅔ of the insertions occurring in predicted open reading frames (ORFs) while the rest of the insertions occurred in the intergenic regions (N=330) (Table 1).

TABLE 1
Percentage and number of unique insertions in a library of
5,060 mutants analyzed

| | | No. of unique | |
|---|---|---|---|
| Insertion Sites | Number | Insertions | % Unique* |
| ORF | 714 | 640 | 89.6 |
| Intergenic region | 436 | 330 | 75.7 |
| Total | 1150 | 970 | 84.3 |

*indicates the percentage of insertions in unique sites within ORF or intergenic regions.

Among the 970 unique insertions within ORFs, only 288 of the predicted mycobacterial ORFs were disrupted at least once by the transposition of Tn5367 indicating that more than an insertion occurred multiple times in some genes. In fact, only 10.4% of disrupted ORFs showed more than one insertion per ORF indicating the presence of “hot spots” for transposition with Tn5367. Compared to insertions in ORFs, a higher rate by at least two times was observed when intergenic regions (24.3%) were examined (Table 2). Overall, the structure of the M. paratuberculosis mutant library was similar to that constructed in M. tuberculosis.

TABLE 2
Characterization of M. paratuberculosis mutants with high insertion
frequency (>10 insertions)

| | Genome | | No. of | | Gene |
|---|---|---|---|---|---|
| | Coordinates | Gene ID | insertions | G + C % | products* |
| Coding | 1297579-1298913 | MAP1235 | 43 | 55.88 | Hypothetical |
| regions | | | | | protein |
| | 1719957-1721030 | MAP 1566 | 42 | 58.19 | Hypothetical |
| | | | | | protein |
| | 878808-880535 | MAP 0856C | 25 | 57.98 | Hypothetical |
| | | | | | protein |
| | 877826-878770 | MAP 0855 | 25 | 59.57 | Hypothetical |
| | | | | | protein |
| | 4266449-4267747 | MAP 3818 | 15 | 59.66 | Hypothetical |
| | | | | | protein |
| | 1295719-1296441 | MAP 1233 | 13 | 50.48 | Hypothetical |
| | | | | | protein |
| | 1296587-1297387 | MAP 1234 | 13 | 57.42 | Hypothetical |
| | | | | | protein |
| | 4803081-4803626 | MAP 4327C | 12 | 60.25 | Hypothetical |
| | | | | | protein |
| | 299412-300203 | MAP 0282C | 11 | 60.47 | Hypothetical |
| | | | | | protein |
| Intergenic | 2380554-2381286 | MAP2149c-MAP2150 | 97 | 54.3 | Hypothetical |
| regions | | | | | proteins |
| | 1276333-1276722 | MAP1216c-MAP1217c | 44 | 52.9 | LpqQ & |
| | | | | | hypothetical |
| | | | | | protein |
| | 1997030-1997898 | MAP1820-MAP1821c | 26 | 54.01 | Hypothetical |
| | | | | | proteins |
| | 4455022-4458337 | MAP3997c-MAP3998c | 21 | 53.9 | SerB and |
| | | | | | hypothetical |
| | | | | | protein |
| | 1409338-1410190 | MAP1318c-MAP1319 | 20 | 57.4 | Adenylate |
| | | | | | cyclase |
| | 2383052-2384295 | MAP2151-MAP2152c | 20 | 54.1 | Hypothetical |
| | | | | | proteins |
| | 300204-301106 | MAP0282c-MAP0283c | 17 | 58.2 | Hypothetical |
| | | | | | proteins |
| | 31518-32640 | MAP0027-MAP0028c | 13 | 57.8 | Hypothetical |
| | | | | | proteins |
| | 4263656-4264948 | MAP3815-MAP3816 | 13 | 60.4 | Hypothetical |
| | | | | | proteins |
| | 4810959-4811624 | MAP4333-MAP4334 | 11 | 56.7 | Hypothetical |
| | | | | | proteins |

*Gene products were described based on cluster of proteins analysis with at least 50% identity to other mycobacterial spp. (http://www.tigr.org/tigr-scripts/CMR2). For intergenic regions, the products of both flanking genes were listed.

More scrutiny of the DNA sequences in both coding and intergenic regions revealed that regions most susceptible to transposon insertions are those with G+C content ranging from 50.5% to 60.5%, which is considerably lower than the average G+C content of the whole M. paratuberculosis (69.2%) (Table 2). Analysis of the flanking regions of Tn5367 site of insertion in genes with high frequency of transposition (N≧4) identified areas of AT or TA repeats (e.g. TTT(T/A), AA(A/T) or TAA) as the most predominant sequences.

To illustrate the randomness of the Tn5367 transposition in M. paratuberculosis genome, the gene positions of all sequenced mutants were mapped to the genome sequence of M. paratuberculosis K10 (GenBank No. AE016958). Additionally, several mutants showed insertion into ORFs that have multiple copies in the genome (e.g. gene families or paralogous genes). These were excluded from further analysis.

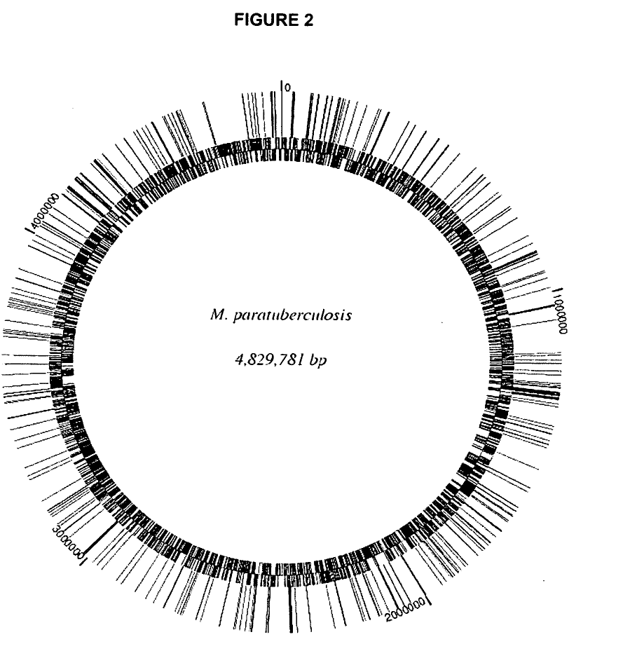

As shown in FIG. 2, the transposition insertions were distributed in all parts of the genome without any apparent bias to a particular area. Overall, 1,128 mutants underwent the second level of bioinformatic analysis. FIG. 2 shows the distribution of 1,128 transposon-insertion sites on the chromosome of M. paratuberculosis K-10 indicated by long bars on the outer-most circle. The inner two circles of short bars show predicted genes transcribed in sense or antisense directions.

To further analyze the expected phenotypes of the disrupted genes, the flanking sequences of each disrupted gene were examined, to determine their participation in transcriptional units such as operons. This analysis could reveal potential polar effect that could be observed in some mutants. Using the operon prediction algorithm (OPERON), approximately 124 (43.0%) of disrupted ORFs were identified as members of 113 putative operons (Table 3), indicating possible phenotypes related to disruption of function encoded by the whole operon and not just the disrupted gene. A total of 52 of the disrupted genes were within the last gene of an operon and were unlikely to affect the expression of other genes.

A total of 23 of the Tn5367 insertions were counted in several genes of the same 12 operons suggesting preference of transpositions throughout these sequences. For example, in the kdp operon (encoding putative potassium translocating proteins), 4 genes were disrupted among the 5 genes constituting this operon. Overall, sequence analysis of transposon junction sites identified disruption of a unique set of genes scattered all over the genome.

TABLE 3
Operon analysis of 288 ORFs disrupted by transposons in this study

| | Operon (%) | Not in operon (%) |
|---|---|---|
| Number | 124 (43.0) | 164 (56.9) |
| First gene | 40 (32.3) | N/A* |
| Middle gene | 32 (35.8) | N/A |
| Last gene | 52 (41.9) | N/A |

*N/A: Not applicable

Sequence Analysis of Disrupted Genes

A total of 288 genes represented by 970 mutants were identified as disrupted from the initial screening of the transposon mutant library constructed in M. paratuberculosis. Examining the potential functional contribution of each disrupted gene among different functional classes encoded in the completely sequenced genome of M. paratuberculosis K10 strain will better characterize their roles in infection. With the help of the Cluster of Orthologous Group website (http://www.ncbi.nlm.nih.gov/COG/), disrupted genes were sorted into functional categories (Table 4). Six genes did not have a match in the COG functional category of M. paratuberculosis and consequently were analyzed using M. tuberculosis functional category (http ://genolist.pasteur.fr/TubercuList/). These genes are involved in different cellular processes such as lipid metabolism (desA1), cell wall biosynthesis (mmpS4) and several possible lipoproteins (IppP, IpqJ, IpqN) including a member of the PE family (PE6).

TABLE 4
List of functional categories of 288 disrupted genes that were identified

| | Coding | | Mutants | |
|---|---|---|---|---|
| | Sequences | | Number | |
| | Number in | | Number | |
| Functional Category | genome | % in genome | mutant | % in genome |
| Translation | 154 | 3.5 | 6 | 3.9 |
| RNA processing and modification | 1 | 0.02 | 0 | 0.0 |
| Transcription | 262 | 6.0 | 8 | 3.1 |
| Replication, recombination and repair | 179 | 4.1 | 13 | 7.3 |
| Chromatin structure and dynamics | 1 | 0.02 | 0 | 0.0 |
| Cell cycle control, mitosis and meiosis | 34 | 0.8 | 3 | 8.8 |
| Defense mechanisms | 46 | 1.1 | 5 | 10.9 |
| Signal transduction mechanisms | 112 | 2.6 | 6 | 5.4 |
| Cell wall/membrane biogenesis | 132 | 3.0 | 12 | 9.1 |
| Cell motility | 10 | 0.2 | 0 | 0.0 |
| Intracellular trafficking and secretion | 20 | 0.5 | 0 | 0.0 |
| Posttranslational modification, protein | 102 | 2.3 | 5 | 4.9 |
| turnover, chaperones | | | | |
| Energy production and conversion | 277 | 6.4 | 10 | 3.6 |
| Carbohydrate transport and metabolism | 187 | 4.3 | 18 | 9.6 |
| Amino acid transport and metabolism | 246 | 5.7 | 16 | 6.5 |
| Nucleotide transport and metabolism | 67 | 1.5 | 2 | 3.0 |
| Coenzyme transport and metabolism | 126 | 2.9 | 3 | 2.4 |
| Lipid transport and metabolism | 326 | 7.5 | 20 | 6.1 |
| Inorganic ion transport and metabolism | 174 | 4.0 | 9 | 5.2 |
| Secondary metabolites biosynthesis, | 357 | 8.2 | 26 | 7.3 |
| transport and catabolism | | | | |
| General function prediction only | 375 | 8.6 | 30 | 8.0 |
| Unknown function | 248 | 5.7 | 16 | 6.5 |
| Unknown | 914 | 21.0 | 80 | 8.8 |

Interestingly, genes involved in cell motility, intracellular trafficking and secretions were not represented in the mutants that were analyzed so far despite their comprising a substantial number of genes (N=30) (Table 4). However, for most functional groups, the percentage of disrupted genes ranged between 3-11% of the genes encoded within the M. paratuberculosis genome.

In most of the functional classes, the percentage of disrupted genes among mutants agreed with the percentage of particular functional class to the rest of the genome. Only 2 gene groups (bacterial defense mechanisms and cell cycling) were over-represented in the mutant library indicating potential sequence divergence from the high G+C content of the rest of the genome, which favorably agreed with the Tn5367 insertional bias discussed before.

Colonization of Transposon Mutants to Mice Organs

To identify novel virulence determinants in M. paratuberculosis, the mouse model of paratuberculosis was employed to characterize selected transposon mutants generated in this study. Bioinformatic analysis was used to identify genes with potential contribution to virulence. Genes were selected if information on their functional role was available, especially genes involved in cellular process believed to be necessary for survival inside the host or genes similar to known virulence factors in other bacteria (Table 5).

The screen for virulence determinants was designed to encompass mutations in a broad range of metabolic pathways to determine whether any could play an essential role for M. paratuberculosis persistence during the infection. Genes involved in carbohydrate metabolism (e.g. gcpE, impA), ion transport and metabolism (e.g. kdpC, trpE2) and cell wall biogenesis (e.g. mmpL10, umaA1) were chosen for further investigating in the mouse model of paratuberculosis, and respective mutants were tested in vivo. Also chosen were: a probable isocitrate lyase (aceAB), a gene involved in mycobactin/exocholin synthesis (mbtH2), a possible conserved lipoprotein (IpqP), as well as putative transcriptional regulators (map0834c and map1634).

TABLE 5
Characterization of transposon mutants tested in the mouse model of
paratuberculosis

| Gene | Insertion %* | Known molecular function |
|---|---|---|
| mmpL10 | 18.6 | Conserved transmembrane transport protein |
| fprA | 56.5 | Adrenodoxin-oxidoreductase |
| papA2 | 12.1 | Conserved polyketide synthase associated |
| | | protein |
| gcpE | 56.8 | Isoprenoid biosynthesis, 4-hydroxy-3- |
| | | methylbut-2-en-1-yl diphosphate synthase |
| papA3_1 | 65.2 | Probable conserved polyketide synthase |
| | | associated protein |
| kdpC | 45.1 | Probable Potassium-transporting ATPase C |
| | | chain |
| umaA1 | 63.5 | Possible mycolic acid synthase |
| pstA | 3.8 | Non-ribosomal binding peptide synthetase |
| fabG2_2 | 70.1 | Putative oxidoreductase activity |
| trpE2 | 81.2 | Probable anthranilate synthase component I |
| impA | 52.0 | Probable inositol-monophosphatase |
| cspB | 63.8 | Small cold shock protein |
| aceAB | 95.5 | Probable isocitrate lyase |
| mbtH2 | 64.6 | mbtH_2 protein family, mycobactin/exocholin |
| | | synthesis |
| IpqP | 1.6 | Possible conserved lipoprotein |
| prrA | 83.6 | Transcriptional regulatory, putative two- |
| | | component system regulator |
| map1634 | 88.8 | Transcription factor activity |
| lipN** | deletion | Lipase, esterase protein |

*Insertion % indicates the percentage from start codon of gene.
**lipN mutant was generated by homologous recombination.

Before animal infection, the growth curve of all mutants in Middlebrook 7H9 broth supplemented with kanamycin was shown to be similar to that of the parent strain. However, most mutants reached an OD₆₀₀=1.0 at 35 days compared to 25 days for the ATCC19698, parent strain, which could be attributed to the presence of kanamycin in the growth media. Once mycobacterial strains reached OD₆₀₀=1 .0, they were appropriately diluted and prepared for intraperitoneal (IP) inoculation of 10⁷-10⁸ CFU/mouse. In each case, the bacterial colonization and the nature of histopathology induced post-challenge were compared to the parent strain of M. paratuberculosis inoculated at similar infectious dose.

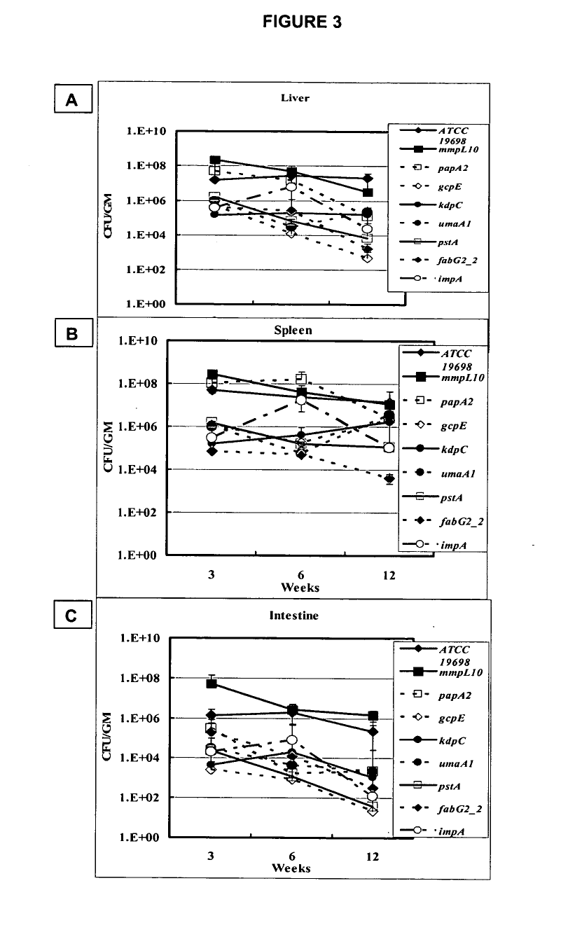

FIG. 3 shows colonization levels of variable M. paratuberculosis strains to mice organs. Groups of mice were infected via intraperitoneal injection (10⁷-10⁸ CFU/mouse) with the wild-type strain (ATCC19698) or one of 11 mutants. Colonization by only 8 mutants is shown in liver (A), spleen (B) and intestine (C) after 3, 6 and 12 weeks post infection. Bars represent the standard errors calculated from the mean of colony counts estimated from organs at different times post infection.

All challenged mice were monitored for 12 weeks post infection with tissue sampling at 3, 6 and 12 weeks post infection. For samples collected at 3 weeks post-infection, only the strains with a disruption in gcpE or kdpC genes displayed significantly (p<0.05) lower colonization levels compared to the parent strain (FIG. 3), especially in the primary target of M. paratuberculosis, the intestine. Some of the mutants (gcpE and kdpc) displayed a significant reduction in the intestinal colony counts starting from 3 weeks post infection and throughout the experiment. At 6 weeks post infection, both papA2 and pstA mutants showed significant colony reduction in the intestine that was maintained in the later time point. At 12 weeks post infection, umaAl, fabG2_{—}2, and impA genes displayed significantly decreased colonization in the intestine (p<0.05) with a reduction of at least 2 logs (FIG. 3C). Colonization levels of the spleen did not show a significant change while levels in the liver and intestine were variable between mutants and wild-type and therefore, they were the most informative organs (FIG. 3).

The four mutants mmpL10, fprA, papA3_{—}1, and trpE2 showed a 10-fold reduction in mycobacterial levels at least in one examined organ by 12 weeks post infection although, this reduction was not statistically significant (p>0.05).

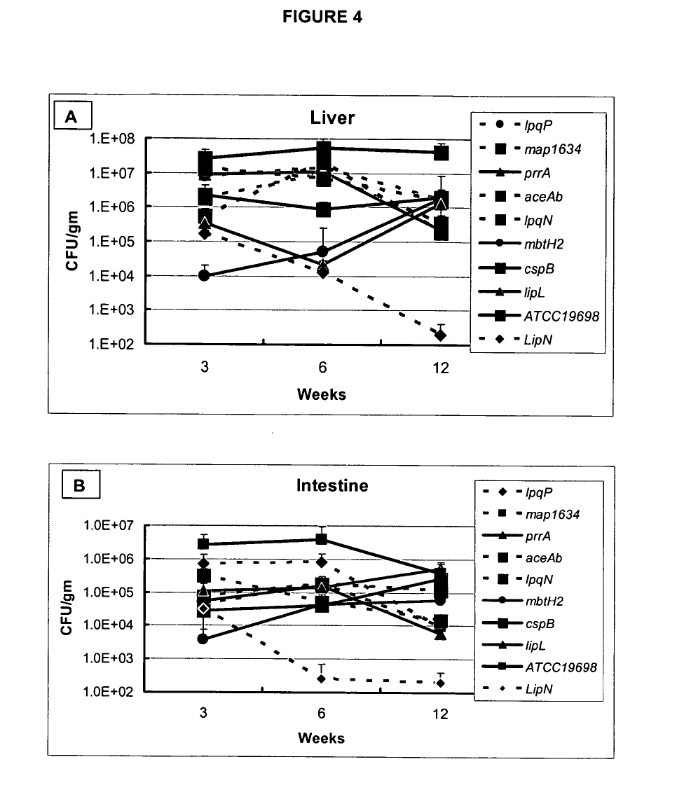

Additional mutants with colonization levels significantly lower in both intestine and liver were identified. Shown in FIG. 4 are data obtained using attenuated mutants with disruption in one of aceAB, mbtH2, IpqP, map0834c, cspB, lipN, or map1634 genes. The graph in FIG. 4A depicts liver colonization of BALB/c mice following infection with 10⁸ CFU/animal of M. paratuberculosis mutants compared to the wild type strain ATCC19698. IP injection was used as a method for infection. Colonization levels in the liver over 3, 6, and 12 weeks post infection were monitored and are shown in FIG. 4A. The graph in FIG. 4B depicts intestinal colonization of BALB/c mice following infection with 10⁸ CFU/animal of M. paratuberculosis mutants compared to the wild type strain ATCC19698. IP injection was used as a method for infection. Colonization levels in the intestine over 3, 6, and 12 weeks post infection were monitored and are shown in FIG. 4B.

Histopathology of Mice Infected with Transposon Mutants

All animal groups infected with mutants or the parent strain displayed a granulomatous inflammatory reaction consistent with infection with M. paratuberculosis using the mouse model of paratuberculosis. Liver sections were the most reflective organ for paratuberculosis where a typical granulomatous response was found. It was exhibited as aggregation of lymphocytes surrounded with a thin layer of fibrous connective tissues.

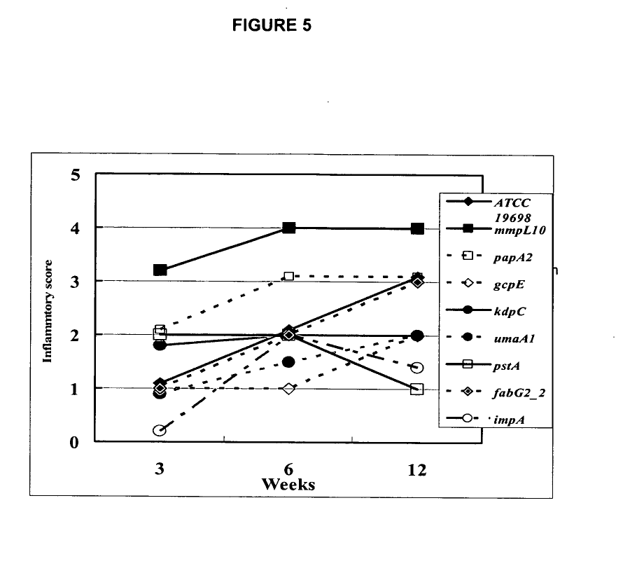

FIG. 5 shows histopathological data from liver of mice infected with M. paratuberculosis strains as outlined in FIG. 3. At 3, 6 and 12 weeks post infection, mice were sacrificed and liver, spleen, and intestine were processed for histopathological examination. Liver sections stained with H&E with arrows indicating granulomatous inflammatory responses were shown in FIG. 4 of U.S. Provisional Patent Application Ser. No. 60/748,852, incorporated herein by reference. FIG. 5 is a chart showing the inflammatory scores of all mice groups.

Granuloma formation was apparent in animals infected with ATCC19698 strain and some mutants such as ΔmmpL10. Both the size and number of granulomas were increased over time indicating the progression of the disease. During early times of infection (3 and 6 weeks sampling), most mutants displayed only lymphocytic inflammatory responses while the formation of granulomas was observed only at the late time (12 weeks samples). Additionally, the severity of inflammation reached level 3 (out of 5) at 12 weeks post-infection for mice infected with ATCC19869 while in the group infected with mutants such as ΔgcpE and ΔkdpC, the granulomatous response was lower (ranged between levels 1 and 2).

When mice infected with ΔmmpL10 were examined, the lymphocyte aggregates were larger in size and were well-separated by fibrous tissues compared to the granuloma formed in mice infected with the ATCC19698. On the other hand, some mutants (e.g. ΔgcpE, ΔimpA) began with relatively minor lesions and remained at this level as time progressed while others (Δpap3_{—}1, fabG2_{—}2) started with mild lesions and progressively increased in severity over time.

A third group of mutants (ΔfprA, ΔkdpC) began with a similar level of response to that of the parent strain and continue to be severely affected until the end of the sampling time.

Generally, by combining the histopathology and colonization data it was possible to assess the overall virulence of the examined mutants and classify disrupted genes into 3 classes. In Class I (early growth mutants), the disruption of genes (e.g. gcpE, KdpC) generated mutants that are not able to multiply efficiently in mice tissues and therefore, a modest level of lesions was generated and their colonization levels were significantly lower than that of wild-type. In Class II (tissue specific mutants), levels of bacterial colonization were significantly reduced in only specific tissues such as umaA1 for liver and papA2 in the intestine at 6 weeks samples. No characteristic pathology of this group could be delineated since only liver sections were reflective of the paratuberculosis using the mouse model employed in this study. In Class III (persistence mutants), levels of colonization were maintained unchanged in the first 6 weeks and then reduced significantly at later times (e.g. fabG2_{—}2 and impA). The lesions formed in animals infected with Class III mutants showed a similar pattern of lesion progression to those of animals infected with the parent strain.

Generally, there was an inverse relationship between granuloma formation scores and mycobacterial colonization levels of mutants for samples collected at 12 weeks post infection. The decline of M. paratuberculosis levels could be attributed to the initiation of a strong immune response represented by an increase of granuloma formation. However, in the case of animals infected with ΔpstA and ΔimpA, the decline of colonization level was consistent with the reduction in granuloma scores.

Overall, large scale characterization of mutant libraries for virulence determinants is shown to be possible, especially when the genome sequence of a given genome is known. The employed approach can be applied in other bacterial systems where there is little information available on pathogen virulence determinants.

Histopathological analyses of mice infected with the five attenuated M. paratuberculosis mutants aceAB, mbtH2, lpqP, map0834c, cspB, lipN, or map1634 showed a decrease in granuloma formation in the liver, compared to the mice infected with the wild type M. paratuberculosis strain ATCC19698.

Characterization of Transposon Mutants

The list of diagnostic targets, i.e., potential virulence determinants disclosed here includes the gcpE gene encoding a product that controls a terminal step of isoprenoid biosynthesis via the mevalonate independent 2-C-methyl-D-erythritol-4-phosphate (MEP) pathway. Because of its conserved nature and divergence from mammalian counterpart, gcpE and its products are considered a suitable target for drug development.

Another diagnostic target, i.e., potential virulence gene, is pstA, which encodes non-ribosomal peptide synthetase in M. tuberculosis with a role in glycopeptidolipids (GPLs) synthesis. The GPLs is a class of species-specific mycobacterial lipids and major constituents of the cell envelopes of many non-tuberculous mycobacteria as well, such as M. smegmatis.

Disruption of umaA1 also resulted in lower colonization levels in all organs examined at 6 weeks post infection and forward.

Additional potential virulence determinants include papA3_{—}1 and papA2, genes that are members of the polyketide synthase associated proteins family of highly conserved genes. Members of the pap family encode virulence-enhancing lipids. Nonetheless, these two mutants displayed different attenuation phenotypes. The papA2 mutant showed significantly lower CFU than the papA3_{—}1 mutant.

The kdpC gene encodes an inducible high affinity potassium uptake system. The kdpC mutant was significantly reduced mostly in the intestinal tissue at early and late stages of infection.

The impA mutant showed significantly reduced levels at late times of infection indicating that impA may possibly play a role in M. paratuberculosis entry into the persistence stage of the infection.

The aceAB mutant showed significantly reduced levels at late times of infection indicating that aceAB may possibly play a role in M. paratuberculosis entry into the persistence stage of the infection. Deletion of a homologue of this gene in M. tuberculosis rendered this mutant attenuated.

The mbtH2 mutant showed significantly reduced levels at early times of infection indicating that mbtH2 may possibly play a role in M. paratuberculosis entry into the intestinal cells or survival in macrophage during early infection. This gene was induced during animal infection using DNA microarrays conducted in the inventor's laboratory.

The lpqP mutant showed significantly reduced levels at late times of infection indicating that lpqP may possibly play a role in M. paratuberculosis entry into the persistence stage of the infection.

The prrA mutant showed significantly reduced levels at late times of infection indicating that prrA may possibly play a role in M. paratuberculosis entry into the persistence stage of the infection. The prrA homologue in M. tuberculosis is two-component transcriptional regulator. This gene was induced at low pH using DNA microarrays conducted in the inventor's laboratory.

The map1634 mutant showed significantly reduced levels at late times of infection indicating that map 1634 may possibly play a role in M. paratuberculosis entry into the persistence stage of the infection. The lipN mutant showed significantly reduced levels at mid and late times of infection indicating that lipN may play an important role in M. paratuberculosis during early and persistent stages of the infection. LipN encodes a lipase which could be important degrading fatty acids. This gene was induced in cow samples using DNA microarrays conducted in the inventor's laboratory.

### Example 2

Bacterial Strains

Mycobacterial isolates (N=34) were collected from different human and domesticated or wildlife animal specimens representing different geographical regions within the USA (Table 6). Mycobacterium avium subsp. paratuberculosis K10 strain, M. avium subsp. avium strain 104 (M. avium 104) and M. intracellulare were obtained from Raul Barletta (University of Nebraska). M. paratuberculosis ATCC19698 and other animal isolates were obtained from the Johne's Testing Center, University of Wisconsin-Madison, while the M. paratuberculosis human isolates were obtained from Saleh Naser (University of Central Florida). All strains were grown in Middlebrook 7H9 broth supplemented with 0.5% glycerol, 0.05% Tween 80 and 10% ADC (2% glucose, 5% BSA fraction V, and 0.85% NaCl) at 37° C. For M. paratuberculosis strains, 2 μg/ml of mycobactin-J (Allied Monitor, Fayette, Mo.) also was added for optimal growth.

TABLE 6
Mycobacterium strains tested in Example 2 of the present invention

| Species | Strain | Host | Sample origin | Location |
|---|---|---|---|---|
| M. avium subsp. | K10 | Cow | Feces | Wisconsin |
| paratuberculosis | ATCC19698 | Cow | Feces | Unknown |
| | JTC33666 | Turkomen markhor (Goat) | Feces | California |
| | JTC33770 | Cow | Feces | Wisconsin |
| | CW303 | Cow | Feces | Wisconsin |
| | 1B | Human | Ileum | Florida |
| | 3B | Human | Ileum | Florida |
| | 4B | Human | Ileum | Florida |
| | 5B | Human | Ileum | Florida |
| | DT3 | British red deer | Feces | Unknown |
| | DT9 | African Eland | Feces | Unknown |
| | DT12 | Chinese Reeve's | Ileum | Unknown |
| | | muntjac (Deer) | | |
| | DT19 | White rhino | Feces | Unknown |
| | JTC1281 | Oryx | Lymph Node | Florida |
| | JTC1282 | Cow | Lymph Node | Wisconsin |
| | JTC1283 | Cow | Feces | Georgia |
| | JTC1285 | Goat | Feces | Virginia |
| | JTC1286 | Cow | Ileum | Wisconsin |
| M. avium subsp. | 104 | Human | Blood | Unknown |
| avium | T93 | Cow | Feces | Texas |
| | T99 | Cow | Feces | Texas |
| | T100 | Cow | Feces | Texas |
| | DT30 | Angolan springbok | Feces | Unknown |
| | DT44 | Formosan Reeve's | Lymph Node | Unknown |
| | | muntjac (Deer) | | |
| | DT78 | Water buffalo | Ileum | Unknown |
| | DT84 | Lowland wisent | Lymph Node | Unknown |
| | DT247 | Cuvier's gazelle | Lymph Node | Unknown |
| | JTC956 | Ankoli | Feces | Florida |
| | JTC981 | Bongo | Feces | Florida |
| | JTC982 | Nyala | Feces | Florida |
| | JTC1161 | Cow | Feces | Florida |
| | JTC1262 | Bison | Lymph Node | Montana |
| | JTC33793 | Dama gazelle | Feces | Indiana |
| M. intracellulare | mc²76 | Human | Sputum | Unknown |

Microarray Design

Oligonucleotide microarrays were synthesized in situ on glass slides using a maskless array synthesizer. Probe sequences were chosen from the complete the genome sequence of M. avium 104. Sequence data of M. avium 104 strain was obtained from The Institute for Genomic Research through the website at http://www.tigr.org. Open reading frames (ORFs) were predicted using GeneMark software. For every ORF, 18 pairs of 24-mer sequences were selected as probes. Each pair of probes consists of a perfect match (PM) probe, along with a mismatch (MM) probe with mutations at the 6th and 12th positions of the corresponding PM probes. A total of ˜185,000 unique probe sequences were synthesized on derivatized glass slides by NimbleGen Systems (Madison, Wis.).

Genomic DNA Extraction and Labeling

Genomic DNA was extracted using a modified CTAB-based protocol followed by two rounds of ethanol precipitation. For each hybridization, 10 μg of genomic DNA was digested with 0.5 U of RQ1 DNase (Promega, Madison, Wis.) until the fragmented DNA was in the range of 50-200 bp (examined on a 2% agarose gel). The reaction was stopped by adding 5 μl of DNase stop solution and incubating at 90° C. for 5 minutes. Digested DNA was purified using YM-10 microfilters (Millipore, Billerica, Mass.).

Genomic DNA hybridizations were prepared by an end-labeling reaction. Biotin was added to purified mycobacterial DNA fragments (10 μg) using terminal deoxynucleotide transferase in the presence of 1 μM of biotin-N6-ddATP at 37° C. for 1 hr. Before hybridization, biotin-labeled gDNA was heated to 95° C. for 5 minutes, followed by 45° C. for 5 minutes, and centrifuged at 14,000 rpm for 10 minutes before adding to the microarray slide.

After microarray hybridization for 12-16 hrs, slides were washed in non-stringent (6×SSPE and 0.01% Tween-20) and stringent (100 mM MES, 0.1 M NaCl, and 0.01% Tween 20) buffers for 5 min each, followed by fluorescent detection by adding Cy3 streptavidin (Amersham Biosciences Corp., Piscataway, N.J.). Washed microarray slides were dried by argon gas and scanned with an Axon GenPix 4000B (Axon Instrument, Union City, Calif.) laser scanner at 5 μm resolution. Replicate microarrays were hybridized for every genome tested. Two hybridizations of the same genomic DNA with high reproducibility (correlation coefficient >0.9) were allowed for downstream analysis.

Data Analysis and Prediction of Genomic Deletions

The images of scanned microarray slides were analyzed using specialized software (NimbleScan) developed by NimbleGen Systems. The average signal intensity of a MM probe was subtracted from that of the corresponding PM probe. The median value of all PM-MM intensities for an ORF was used to represent the signal intensity for the ORF. The median intensities value for each slide was normalized by multiplying each signal by a scaling factor that was 1000 divided by the average of all median intensities for that array.

To compare hybridization signals generated from each of the genomes to that of M. avium 104, the normalized data from replicate hybridizations were exported to R language program with the EBarrays package version 1.1, which employs a Bayesian statistical model for pair-wise genomic comparisons using a log-normal-normal model. Genes with the probability of differential expression larger than 0.5 were considered significantly different between the genomes of M. avium and M. paratuberculosis.

The hybridization signals corresponding to each gene of all investigated genomes were plotted according to genomic location of M. avium 104 strain using the GenVision software (DNAStar Inc., Madison, Wis.). The same data set was also analyzed by MultiExperiment Viewer 3.0 to identify common cluster patterns among mycobacterial isolates.

Microarray Analysis of M. avium and M. paratuberculosis Genomes

Genomic rearrangements among M. avium and M. paratuberculosis isolated from variable hosts were investigated, to identify diagnostic targets for microbial infection. The analysis began using 5 mycobacterial isolates employing DNA microarrays and was expanded to include an additional 29 isolates employing a more affordable technology of PCR followed by direct sequencing. All of the isolates were collected from human and domesticated or wildlife animal sources and had been previously identified at the time of isolation using standard culturing techniques for M. avium and M. paratuberculosis. The identity of each isolate was confirmed further by acid-fast staining and positive PCR amplification of IS900 sequences from all M. paratuberculosis. Additionally, the growth of all M. paratuberculosis isolates were mycobactin-J dependent while all M. avium isolates were not.

Before starting the microarray analysis, an hsp65 PCR typing protocol was performed to ensure the identity of each isolate. The PCR typing protocol agreed with earlier characterization of all mycobacterial isolates used throughout this study. FIG. 5A of U.S. Provisional Patent Application Ser. No. 60/748,852, incorporated by reference, depicts the PCR confirmation of the identity of the examined genomes.

To investigate the extent of variation among M. avium and M. paratuberculosis on a genome-wide scale, oligonucleotide microarrays were designed from the M. avium 104 strain genome sequence. The GeneMark algorithm was used to predict potential ORFs in the raw sequence of M. avium genome obtained from TIGR. A total of 4987 ORFs were predicted for M. avium compared to 4350 ORFs predicted in M. paratuberculosis. Relaxed criteria for assigning ORFs were chosen (at least 100 bp in length with a maximal permitted overlap of 30 bases between ORFs) to use a comprehensive representation of the genome to construct DNA microarrays.

Similar to other bacterial genomes, the average ORF length was ˜1 Kb. Using the ASAP comparative genomic software suite, the ORFs shared by M. paratuberculosis and M. avium had an average percent identity of 98%, a result corroborated by others. BLAST analysis of the ORFs from both genomes show that about 65% (N=2557) of the genes have a significant match (E<10-10) in the other genome.

To test the reliability of genomic DNA extraction protocols and microarray hybridizations, the signal intensities of replicate hybridizations of the same mycobacterial genomic DNA were compared using scatter plots. ORFs with positive hybridization signals in at least 10 probe pairs were normalized and used for downstream analysis to ensure the inclusion of only ORFs with reliable signals. In all replicates, independently isolated hybridized samples of gDNA had high correlation coefficients (r>0.9).

To investigate the genomic relatedness among isolates compared to the M. avium 104 strain, a hierarchical cluster analysis was used to assess the similarity of the hybridization signals among isolates on a genome-wide level. FIG. 5C of U.S. Provisional Patent Application Ser. No. 60/748,852, incorporated by reference, shows a dendogram displaying the overall genomic hybridization signals generated from biological replicates of different mycobacterial isolates from animal or human (HU) sources.

Within the M. paratuberculosis cluster, the human and the clinical animal isolates were highly similar to each other than to the ATCC19698 reference strain, implying a closer relatedness between human and clinical isolate of M. paratuberculosis. Interestingly, despite the high degree of similarity between genes shared among isolates, hundreds of genes appeared to be missing from different genomes relative to M. avium genome. Most of the genes were found in clusters in the M. avium 104 genome, the reference strain used for designing the microarray chip. Consequently, regions absent in M. avium 104 but present in other genomes could not be identified in this analysis.

PCR Verification and Sequence Analysis

To confirm the results predicted by microarray hybridizations, a 3-primer PCR protocol was used to amplify the regions flanking predicted genomic islands. For every island, one pair of primers (F—forward and R1—reverse 1) was designed upstream of the target region and a third primer (R2-reverse 2) was designed downstream of the same region. The primers were designed so that expected lengths of the products were less than 1.5 Kb between F and R1 and less than 3 Kb between F and R2 when amplified from the genomes with the deleted island. Each PCR contained 1 M betaine, 50 mM potassium glutamate, 10 mM Tris-HCl pH 8.8, 0.1% of Triton X-100, 2 mM of magnesium chloride, 0.2 mM dNTPs, 0.5 μM of each primer, 1 U Taq DNA polymerase and 15 ng genomic DNA. The PCR cycling condition was 94° C. for 5 minutes, followed by 30 cycles of 94° C. for 1 minute, 59° C. for 1 minute and 72° C. for 3 minutes.

All PCR products were examined using 1.5% agarose gels and stained with ethidium bromide. To further confirm sequence deletions, amplicons flanking deleted regions were sequenced using standard BigDye® Terminator v3.1 (Applied Biosystems, Foster City, Calif.) and compared to the genome sequence of M. paratuberculosis or M. avium using BLAST alignments.

Large Genomic Deletions Among M. avium and M. paratuberculosis Isolates

To better analyze the hybridization signals generated from examined genomes, a Bayesian statistical principle (EBarrays package) was used to compare the hybridization signals generated from different isolates relative to the signals generated from M. avium 104 genome. The Bayesian analysis estimates the likelihood of observed differences in ORF signals for each gene between each isolate and the M. avium 104 reference strain.

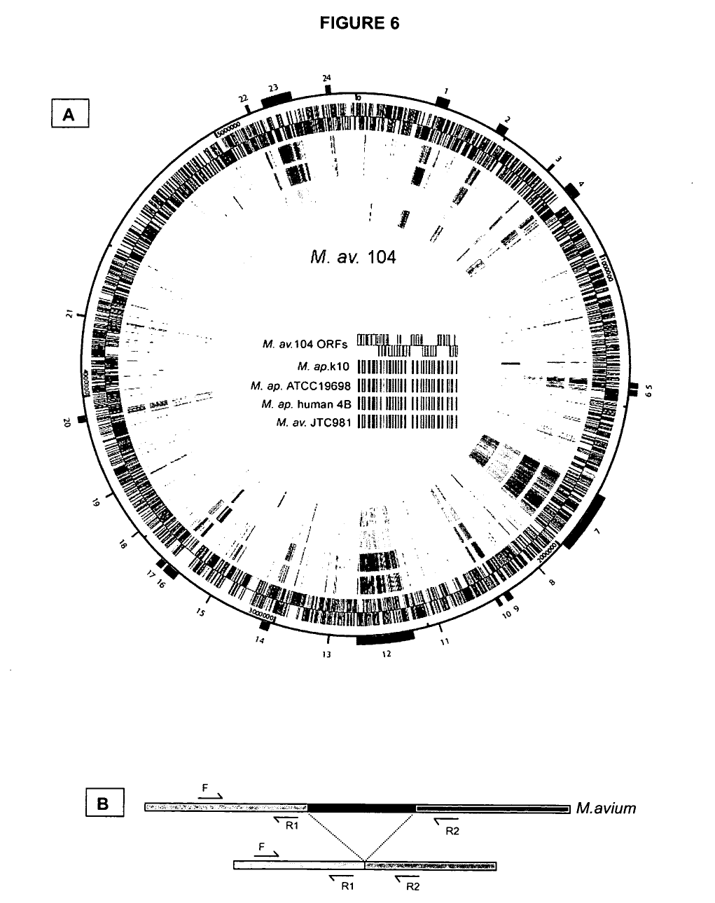

FIG. 6A depicts a genome map based on M. avium sequence displaying GIs deleted in the examined strains as predicted by DNA microarrays. Inner circles denote the microarray hybridization signals for each examined genome (see legend in center). The outermost dark boxes denote the location of all GIs associated with M. avium. A large number of differences were seen among isolates, including many ORFs scattered throughout the genome.

PCR and sequencing were used to confirm deletions identified by microarrays. FIG. 6B depicts a diagram illustrating the PCR and sequence-based strategy implemented to verify the genomic deletions. Three primers for each island were designed including a forward (F) and 2 reverse primers. When regions included 3 or more consecutive ORFs, they were defined as a genomic island (GI) regardless of the size. Applying such criterion for genomic islands (GIs), 24 islands were present in M. avium 104 but absent from all M. paratuberculosis isolates, regardless of the source of the M. paratuberculosis isolates (animal or human). The GIs ranged in size from 3 to 196 Kb (Table 7) with a total of 846 Kb encoding 759 ORFS. Interestingly, a clinical strain of M. avium (JTC981) was also missing 7 GIs (nearly 518 Kb) in common with all M. paratuberculosis isolates, in addition to the partial absence of 5 other GIs. This variability indicated a wide-spectrum of genomic diversity among M. avium strains that was not evident among M. paratuberculosis isolates.

To confirm the absence of GI regions from isolates, a strategy based on PCR amplification of the flanking regions of each GI was used, followed by sequence analysis to confirm the missing elements. Because the size of most of the genomic island regions exceeds the length of the amplification capability of a typical PCR reaction, 3 primers for each island were designed, including one forward and 2 reverse primers (FIG. 6B). This strategy was successfully applied on 21 genomic islands, while amplification from the rest of the islands (N=3) was not possible due to extensive genomic rearrangements.

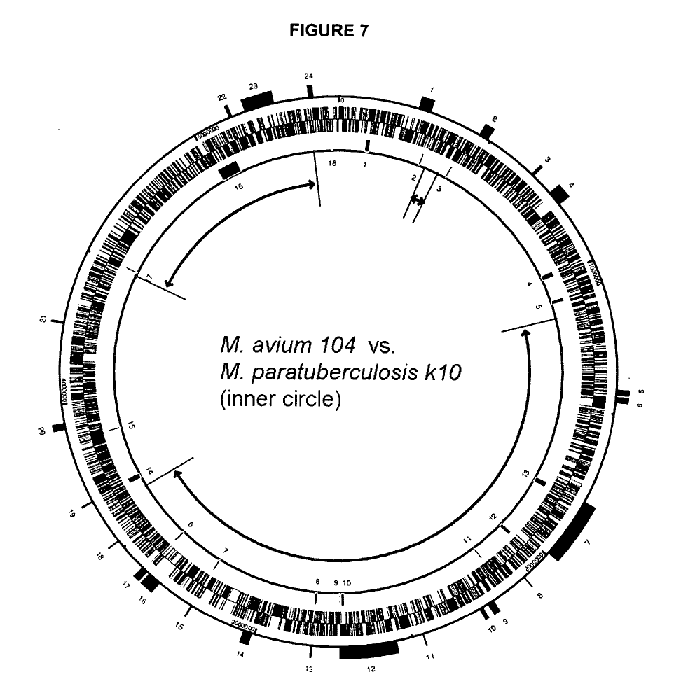

FIG. 7 depicts the synteny of M. avium and M. paratuberculosis genomes.

PCR confirmation of genomic deletions was performed, as shown in FIGS. 8 and 9 of U.S. Provisional Patent Application Ser. No. 60/748,852, incorporated herein by reference. Overall, the PCR and sequencing verified the GI content as predicted by comparative genomic hybridizations (Table 7). The success of this strategy in identifying island deletions provided a protocol to examine several clinical isolates that could not be otherwise analyzed by costly DNA microarrays.

TABLE 7
List of genomic regions that displayed different hybridization signals
using DNA microarrays designed from the genome of M. avium 104 strain

| | | | | | | | PCR and |
|---|---|---|---|---|---|---|---|
| Island | | | M. parat. | M. parat. | M. parat. | M. avium | sequence |
| Number | Start (bp)^{a} | End (bp)^{a} | K10^{b} | 19698 | human | JTC981 | confirmation^{c} |
| 1 | 254,394 | 294,226 | − | − | − | − | Yes |
| 2 | 461,414 | 492,800 | − | − | − | − | Yes |
| 3 | 666,033 | 675,725 | − | − | − | − | Yes |
| 4 | 747,095 | 794,450 | − | − | − | − | Yes |
| 5 | 1,421,722 | 1,439,626 | − | − | − | + | Yes |
| 6 | 1,444,205 | 1,463,365 | − | − | − | + | Yes |
| 7 | 1,795,281 | 1,991,691 | − | − | − | +/− | Yes |
| 8 | 2,097,907 | 2,100,883 | − | − | − | − | Yes |
| 9 | 2,220,320 | 2,241,163 | − | − | − | +/− | Yes |
| 10 | 2,259,120 | 2,271,610 | − | − | − | − | Yes |
| 11 | 2,462,693 | 2,466,285 | − | − | − | + | Yes |
| 12 | 2,549,555 | 2,730,999 | − | − | − | − | ND |
| 13 | 2,815,625 | 2,821,149 | − | − | − | + | Yes |
| 14 | 3,008,716 | 3,036,980 | − | − | − | + | Yes |
| 15 | 3,214,820 | 3,219,550 | − | − | − | + | ND |
| 16 | 3,340,393 | 3,384,549 | − | − | − | + | Yes |
| 17 | 3,392,586 | 3,413,804 | − | − | − | + | ND |
| 18 | 3,523,417 | 3,527,334 | − | − | − | +/− | Yes |
| 19 | 3,670,518 | 3,675,686 | − | − | − | + | Yes |
| 20 | 3,917,752 | 3,939,034 | − | − | − | +/− | Yes |
| 21 | 4,254,594 | 4,261,488 | − | − | − | +/− | Yes |
| 22 | 5,122,371 | 5,132,301 | − | − | − | + | Yes |
| 23 | 5,174,641 | 5,270,187 | − | − | − | + | Yes |
| 24 | 5,378,903 | 5,395,102 | − | − | − | + | Yes |

^{a}Coordinates of start and end of island based on the genome sequence of M. avium strain 104.
^{b}+ or − denotes presence or absence of genomic regions in examined genomes while +/− denotes incomplete deletion.
^{c}ND—not done.

Bioinformatic Analysis of Genomic Islands

Pair-wise BLAST analysis of the genome sequences of M. avium 104 and M. paratuberculosis K10 was used to further refine the ability to detect genomic rearrangements, especially for regions present in M. paratuberculosis K10 genome but deleted from M. avium 104 genome. The pair-wise comparison allowed to better analyze the flanking sequences for each GI and to characterize the mechanism of genomic rearrangements among examined strains.

BLAST analysis (E scores >0.001 and <25% sequence alignment between ORFs) correctly identified the deleted GIs where ORFs of M. avium were missing in M. paratuberculosis detected by using the comparative genomic hybridization protocol. A large proportion of ORFs in each genome (>75%) are likely orthologous (>25% sequence alignment of the ORF length and >90% sequence identity at nucleotide level). This high degree of similarity between orthologues indicates a fairly recent ancestor. Looking for consecutive ORFs from M. paratuberculosis that do not have a BLAST match in M. avium identified sets of ORFs representing 18 GIs comprising 240 Kb that are present only in M. paratuberculosis genome (Table 8).

Genes encoded within M. avium and M. paratuberculosis specific islands were analyzed by BLASTP algorithm against the GenPept database (October 19, 2004 release) to identify their potential functions. The BLAST results allowed the assignment of signature features to each island. As detailed in Tables 8 and 9, with the presence of a large number of ORFs encoding mobile genetic elements (e.g. insertion sequences and prophages), several ORFs encode transcriptional regulatory elements, especially from TetR-family of regulators. The polymorphism in TetR regulators could be attributed to their sequences allowing them to be amenable for rearrangements. Alternatively, it is possible that the bacteria are able to differentially acquire specific groups of genes suitable for a particular microenvironment.

Further analysis of the GIs identified islands in both M. avium and M. paratuberculosis (such as MAV-7, MAV-12 and MAP-13) encoding different operons of the mce (mammalian cell entry) sequences that were shown to participate in the pathogenesis of M. tuberculosis. Another island (MAV-17) encodes the drrAB operon for antibiotic resistance, which is a well-documented problem for treating M. avium infection in HIV patients. The GC % of the majority of M. paratuberculosis specific islands (11/18) was at least 5% less than the average GC % of the M. paratuberculosis genome (69%) compared to only 3 GIs (out of 24) specific for M. avium genome (Table 9) with lower than average GC %.

TABLE 8
M. paratuberculosis-specific (MAP) genomic islands deleted in M. avium
genome

| Island | No. of | | Island | | |
|---|---|---|---|---|---|
| Number | ORFs | GC % | Type | Size (bp) | Signature Features |
| MAP-1 | 17 | 63.90 | I | 19,343 | Transposition and TetR-family |
| | | | | | transcriptional regulator genes |
| MAP-2 | 3 | 60.43 | I | 3,858 | Conserved hypothetical proteins |
| MAP-3 | 3 | 66.16 | I | 2,915 | Formate dehydrogenase alpha |
| | | | | | subunit |
| MAP-4 | 17 | 60.66 | I | 16,681 | Transposition, unknown genes and |
| | | | | | a possible prophage |
| MAP-5 | 12 | 69.56 | I | 14,191 | Transposition and oxidoreductase |
| | | | | | genes, PPE family domain protein |
| MAP-6 | 6 | 57.73 | II | 8,971 | Variable genes such as drrC |
| MAP-7 | 6 | 67.26 | II | 6,914 | Transcriptional regulator psrA and |
| | | | | | biosynthesis genes |
| MAP-8 | 8 | 61.59 | II | 7,915 | TetR-family transcriptional regulator |
| | | | | | and unknown genes |
| MAP-9 | 10 | 65.49 | II | 11,202 | Transposition, metabolic and TetR- |
| | | | | | family transcriptional regulator |
| | | | | | genes |
| MAP-10 | 3 | 66.68 | II | 2993 | Biosynthesis of cofactors, prosthetic |
| | | | | | groups, and carriers transcriptional |
| | | | | | regulator, TetR family domain |
| | | | | | protein |
| MAP-11 | 4 | 62.89 | I | 2,989 | Serine/threonine protein kinase and |
| | | | | | glyoxalase genes |
| MAP-12 | 11 | 61.08 | I | 11,977 | Transposition, iron metabolism |
| | | | | | genes and a prophage |
| MAP-13 | 19 | 66.01 | II | 19,977 | TetR-family transcript, regulator and |
| | | | | | mce family proteins |
| MAP-14 | 19 | 65.76 | II | 19,315 | Possible prophage and unknown |
| | | | | | proteins |
| MAP-15 | 3 | 62.93 | I | 4,143 | Unknown proteins and a prophage |
| | | | | | function genes |
| MAP-16 | 56 | 64.32 | I | 79,790 | Transposition and iron regulatory |
| | | | | | genes |
| MAP-17 | 5 | 61.60 | I | 3,655 | Unknown proteins and a multi-copy |
| | | | | | phage resistance gene |
| MAP-18 | 3 | 60.36 | I | 3,512 | Hypothetical proteins |
| Total | 204 | | | 239,969 | |

TABLE 9
Characteristics of M. avium-specific (MAV) genomic islands

| Island | No. of | | Island | Size | |
|---|---|---|---|---|---|
| Number | ORFs | GC % | Type | (bp) | Signature Features |
| MAV-1 | 38 | 68.93 | I | 39,833 | Eukaryotic genes with an integrase |
| | | | | | gene |
| MAV-2 | 32 | 65.87 | I | 31,387 | Transposition and M. tuberculosis |
| | | | | | genes |
| MAV-3 | 10 | 63.34 | I | 9,693 | Insertion sequence and M. tuberculosis |
| | | | | | or M. avium genes |
| MAV-4 | 53 | 66.83 | I | 47,356 | PPE family and eukaryotic genes |
| MAV-5 | 16 | 64.10 | I | 17,905 | Transposition and insertion |
| | | | | | sequences genes |
| MAV-6 | 23 | 68.80 | I | 19,161 | Transposition, transcript. regulator |
| | | | | | and heavy metal resistance genes |
| MAV-7 | 187 | 65.50 | II | 196,411 | Transposition, transcript. regulators, |
| | | | | | cell entry, iron regulation genes |
| MAV-8 | 3 | 65.18 | I | 2,977 | Transposition and transcriptional |
| | | | | | regulator genes |
| MAV-9 | 15 | 62.43 | I | 20,844 | Transposition and type III restriction |
| | | | | | system endonuclease genes |
| MAV-10 | 12 | 63.87 | I | 12,491 | Transposition genes |
| MAV-11 | 5 | 65.45 | I | 3,593 | Reductases and hypothetical proteins |
| MAV-12 | 168 | 65.05 | II | 181,445 | Transposition, transcriptional |
| | | | | | regulators and cell entry genes |
| MAV-13 | 7 | 67.78 | II | 5,525 | Transcriptional regulator |
| MAV-14 | 26 | 67.32 | I | 28,265 | Transposition and M. tuberculosis |
| | | | | | genes |
| MAV-15 | 3 | 64.12 | II | 4,731 | Streptomyces and M. leprae genes |
| MAV-16 | 6 | 69.64 | I | 44,157 | Transposition and Pst genes |
| MAV-17 | 20 | 65.23 | II | 21,219 | Transposition and drrAB genes |
| | | | | | (antibiotic resistance) |
| MAV-18 | 4 | 68.13 | I | 3,918 | Transcriptional regulator and |
| | | | | | Streptomyces genes |
| MAV-19 | 4 | 65.30 | I | 5,169 | Transposition genes |
| MAV-20 | 15 | 63.93 | I | 21,283 | Transposition, transcriptional |
| | | | | | regulator and membrane-protein |
| | | | | | genes of M. tuberculosis |
| MAV-21 | 8 | 65.93 | I | 6,895 | Transposition and antigen genes |
| MAV-22 | 9 | 67.71 | I | 9,931 | Transcriptional regulator and |
| | | | | | metalloprotease genes |
| MAV-23 | 77 | 64.08 | I | 95,547 | Transposition, transcript. regulators, |
| | | | | | secreted proteins, cell entry genes |
| MAV-24 | 18 | 70.25 | I | 16,200 | Hypothetical and unknown proteins |
| | | | | | from M. tuberculosis and |
| | | | | | Streptomyces |
| Total | 759 | | | 845,936 | |

Genomic Deletions Among Field Isolates of M. avium

Microarrays and PCR analysis of 5 mycobacterial isolates identified the presence of variable GIs between M. avium and M. paratuberculosis genomes. To analyze the extent of such variations among clinical isolates circulating in both human and animal populations, PCR and a sequencing-based strategy were used to examine 28 additional M. avium and M. paratuberculosis isolates collected from different geographical locations within the USA (Table 6). An additional isolate of M. intracellulare was included as a representative strain that belongs to the MAC group but not a subspecies of M. avium.

For PCR amplification, GIs spatially scattered throughout the M. avium and M. paratuberculosis genomes were examined (Tables 10, 11) to identify any potential rearrangements in all quarters of the genome. Because of the wide-spectrum diversity observed among M. avium genomes, 4 GIs (MAV-3, 11, 21 and 23) were chosen to assess genomic rearrangements in clinical isolates. Because of the limited diversity observed among M. paratuberculosis genomes, a total of 6 M. paratuberculosis-specific GIs (MAP-1, 3, 5, 12, 16 and 17) were chosen for testing genomic rearrangements. As suggested from the initial comparative genomic hybridizations, clinical isolates of M. paratuberculosis showed a limited diversity in the existence of M. avium-specific islands (DT9 clinical isolate from a red deer) indicating the clonal nature of this organism (Table 10).

To the contrary, M. avium isolates showed a different profile from both M. avium 104 and M. avium JTC981 indicating extensive variability within M. avium isolates. A similar pattern of genomic rearrangements was observed when M. paratuberculosis-specific GIs were analyzed using M. avium and M. paratuberculosis isolates (Table 11). Most of the M. paratuberculosis clinical isolates with deleted GIs were from wildlife animals suggesting that strains circulating in wildlife animals could provide a potential source for genomic rearrangements in M. paratuberculosis.

TABLE 10
PCR identification of selected MAV-island regions from 29 clinical
isolates of M. paratuberculosis and M. avium collected from different
states

| | | Genomic island | | | |
|---|---|---|---|---|---|
| Clinical | | | MAV- | MAV- | |
| Isolate | Subspecies | MAV-3 | 11 | 21 | MAV-23 |
| JTC33666 | M. paratuberculosis | − | − | − | − |
| JTC33770 | M. paratuberculosis | − | − | − | − |
| CW303 | M. paratuberculosis | − | − | − | − |
| 1B | M. paratuberculosis | − | − | − | − |
| 3B | M. paratuberculosis | − | − | − | − |
| 4B | M. paratuberculosis | − | − | − | − |
| 5B | M. paratuberculosis | − | − | − | − |
| DT3 | M. paratuberculosis | − | − | − | − |
| DT9 | M. paratuberculosis | + | N/A | − | − |
| DT12 | M. paratuberculosis | − | − | − | − |
| DT19 | M. paratuberculosis | − | − | − | − |
| JTC1281 | M. paratuberculosis | − | − | − | − |
| JTC1282 | M. paratuberculosis | − | − | − | − |
| JTC1283 | M. paratuberculosis | − | − | − | − |
| JTC1285 | M. paratuberculosis | − | − | − | − |
| JTC1286 | M. paratuberculosis | − | − | − | − |
| T93 | M. avium | + | − | − | − |
| T99 | M. avium | + | − | − | − |
| T100 | M. avium | + | + | − | − |
| DT30 | M. avium | − | + | + | + |
| DT44 | M. avium | − | + | + | + |
| DT78 | M. avium | − | + | + | + |
| DT84 | M. avium | − | + | − | + |
| DT247 | M. avium | − | + | + | + |
| JTC956 | M. avium | N/A | N/A | N/A | − |
| JTC982 | M. avium | N/A | + | N/A | + |
| JTC1161 | M. avium | + | + | − | − |
| JTC1262 | M. avium | + | − | − | − |
| JTC33793 | M. avium | + | + | + | + |

Symbols (+ or −) denote presence or absence of genomic regions;
N/A denotes no amplification of DNA fragments.

Combined with the hierarchical cluster analysis employed on the whole genome hybridizations, PCR and sequence analyses provided more evidence that genomic diversity is quite extensive among M. avium strains but much less limited in M. paratuberculosis.

As shown in FIG. 10 of U.S. Provisional Patent Application Ser. No. 60/748,852, incorporated herein by reference, PCR analysis was successfully used to establish the distribution of M. paratuberculosis-specific island #1 (MAP-1) within 21 clinical isolates of M. avium and M. paratuberculosis.

Large DNA Fragment Inversions within the Genomes of M. avium Subspecies.

Because of the high similarity among the genomes of M. paratuberculosis and M. avium reported earlier, considerable conservation in the synteny between genomes (gene order) within M. avium strains was expected. The order of GIs was used as markers for testing the conserved gene order and the overall genome structure between M. paratuberculosis and M. avium genomes.

It was unexpectedly discovered that, when the GIs associated with both genomes were aligned, three large genomic fragments in M. paratuberculosis were identified as inverted relative to the corresponding genomic fragments in M. avium. These fragments had the sizes of approximately 1969.4 Kb, 863.8 Kb, and 54.9 Kb (FIG. 7). The largest inverted region (INV-1) of approximately 1969.4 Kb is flanked by MAV-4 and MAV-19. INV-1 encompasses bases 075033 through 3044433 of the M. paratuberculosis genomic sequence. The second inverted region (INV-2) of approximately 863.8 Kb is flanked by MAV-21 and MAV-24. Located near the origin of replication, INV-2 encompasses bases 3885218 through 4748979 of the M. paratuberculosis genomic sequence. The smallest inverted region (INV-3) of approximately 54.9 Kb is flanked by MAV-1 and MAV-2. INV-3 encompasses bases 320484 through 377132 of the M. paratuberculosis genomic sequence.

Because the sequences of the inverted regions and of the flanking MAVs are known, it is possible to use the junction regions (sequences) to identify the presence of either M. paratuberculosis or M. avium in a sample. For example, using the right sets of primers, one skilled in the art would know to detect sequences that are specific to the junction regions that are characteristic for either M. avium or M. paratuberculosis.

Referring to FIG. 7, the location of genomic islands present in M. avium (dark grey boxes) or in M. paratuberculosis (light grey boxes) genomes are drawn to scale on the circular map of M. avium (outer circle) as well as the map of M. paratuberculosis (inner circle). The sequences of M. paratuberculosis K10 (query sequence) compared with the whole genome sequence M. avium 104 ORFs (target sequence) using BLAST algorithm with cut off values of E>0.001 and alignment percentage <25% of the whole gene were accepted as indications for gene deletion. The numerous short bars represent predicted ORFs in forward (outermost) or reverse (innermost) orientations. Large arrows indicate sites of genomic inversions.

Because the bioinformatics analysis used raw genome sequences, PCR and sequencing approach were used to substantiate the genomic inversions in 7 mycobacterial isolates (3 isolates of M. avium and 4 isolates of M. paratuberculosis). As predicted from the initial sequence analysis, primers flanking the junction sites of the inverted regions gave the correct DNA fragment sizes and orientations consistent with the sequence of M. avium and M. paratuberculosis genomes.

TABLE 11
PCR identification of selected MAP-island regions from 29 clinical
isolates of M. paratuberculosis and M. avium collected from different states

| Clinical | | Genomic island | | | | | |
|---|---|---|---|---|---|---|---|
| Isolate | Subspecies | MAP-1 | MAP-3 | MAP-5 | MAP-12 | MAP-16 | MAP-17 |
| JTC33666 | M. paratub. | + | + | + | + | + | + |
| JTC33770 | M. paratub. | + | + | + | + | + | + |
| CW303 | M. paratub. | + | + | + | + | + | + |
| 1B | M. paratub. | + | + | + | + | + | + |
| 3B | M. paratub. | + | + | + | + | + | + |
| 4B | M. paratub. | + | + | + | + | + | + |
| 5B | M. paratub. | + | + | + | + | + | + |
| DT3 | M. paratub. | − | + | + | + | + | + |
| DT9 | M. paratub. | − | + | + | + | + | + |
| DT12 | M. paratub. | + | + | + | + | + | + |
| DT19 | M. paratub. | + | + | + | + | + | + |
| JTC1281 | M. paratub. | − | + | + | + | + | + |
| JTC1282 | M. paratub. | − | + | + | + | + | + |
| JTC1283 | M. paratub. | − | + | + | + | + | + |
| JTC1285 | M. paratub. | − | − | + | + | + | − |
| JTC1286 | M. paratub. | + | + | + | + | + | + |
| T93 | M. avium | − | − | − | − | − | − |
| T99 | M. avium | − | N/A | + | − | + | + |
| T100 | M. avium | + | N/A | + | + | − | + |
| DT30 | M. avium | − | − | − | − | − | − |
| DT44 | M. avium | − | − | − | − | − | − |
| DT78 | M. avium | − | − | + | − | − | + |
| DT84 | M. avium | − | − | − | − | − | − |
| DT247 | M. avium | − | − | + | − | − | − |
| JTC956 | M. avium | N/A | − | N/A | − | + | + |
| JTC982 | M. avium | − | − | + | − | − | − |
| JTC1161 | M. avium | − | − | + | N/A | + | + |
| JTC1262 | M. avium | − | − | − | − | − | − |
| JTC33793 | M. avium | − | − | − | − | − | − |

Symbols (+ or −) denote presence or absence of genomic regions;
N/A denotes no amplification of DNA fragments.

It is to be understood that this invention is not limited to the particular devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters normally encountered in clinical prevention and therapy, obvious to those skilled in the art, are within the scope of this invention. All publications, patents, and patent applications cited herein are incorporated by reference in their entirety for all purposes.

What is claimed is:

1. An isolated MAP genomic island from M. paratuberculosis.

2. The isolated MAP genomic island of claim 1 further comprising a label.

3. The MAP genomic island of claim 1 wherein the MAP genomic island is any one of MAP-1, MAP-2, MAP-3, MAP-4, MAP-5, MAP-6, MAP-7, MAP-8, MAP-9, MAP-10, MAP-11, MAP-12, MAP-13, MAP-14, MAP-15, MAP-16, MAP-17, or MAP-18, or homologs thereof.

4. An isolated MAV genomic island from M. avium.

5. The isolated MAV genomic island of claim 4 further comprising a label.

6. The MAV genomic island of claim 4 wherein the MAV genomic island is any one of MAV-1, MAV-2, MAV-3, MAV-4, MAV-5, MAV-6, MAV-7, MAV-8, MAV-9, MAV-10, MAV-11, MAV-12, MAV-13, MAV-14, MAV-15, MAV-16, MAV-17, MAV-18, MAV-19, MAV-20, MAV-21, MAV-22, MAV-23, or MAV-24, or homologs thereof.

7. A nucleic acid probe sequence comprising a nucleic acid sequence having at least 70% homology with any contiguous nucleotide sequence of at least 20 nucleotides that are substantially identical to the target sequence comprising at least one of:
a) gcpE, pstA, kdpC, papA2, impA, umaA1, fabG2_{—}2, aceAB, mbtH2, lpqP, map0834c, cspB, lipN, or map1634 genes of M. paratuberculosis;
b) MAP-1, MAP-2, MAP-3, MAP-4, MAP-5, MAP-6, MAP-7, MAP-8, MAP-9, MAP-10, MAP-11, MAP-12, MAP-13, MAP-14, MAP-15, MAP-16, MAP-17, or MAP-18;
c) MAV-1, MAV-2, MAV-3, MAV-4, MAV-5, MAV-6, MAV-7, MAV-8, MAV-9, MAV-10, MAV-11, MAV-12, MAV-13, MAV-14, MAV-15, MAV-16, MAV-17, MAV-18, MAV-19, MAV-20, MAV-21, MAV-22, MAV-23, or MAV-24; or
d) a junction sequence between an inverted genomic fragment INV and a flanking genomic island;
or homologs thereof.

8. The nucleic acid probe sequence of claim 7 further comprising a label.

9. A method for detecting the presence or absence of a mycobacterial strain or phenotype in a test sample, the method comprising:
a) contacting a probe with a test sample, wherein the probe comprises a nucleic acid sequence having at least 70% homology with any contiguous nucleotide sequence of at least 20 nucleotides that are substantially identical to the target sequence comprising at least one of:
i. gcpE, pstA, kdpC, papA2, impA, umaA1, fabG2_{—}2, aceAB, mbtH2, IpqP, map0834c, cspB, lipN, or map 1634 genes of M. paratuberculosis;
ii. MAP-1, MAP-2, MAP-3, MAP-4, MAP-5, MAP-6, MAP-7, MAP-8, MAP-9, MAP-10, MAP-11, MAP-12, MAP-13, MAP-14, MAP-15, MAP-16, MAP-17, or MAP-18;
iii. MAV-1, MAV-2, MAV-3, MAV-4, MAV-5, MAV-6, MAV-7, MAV-8, MAV-9, MAV-10, MAV-11, MAV-12, MAV-13, MAV-14, MAV-15, MAV-16, MAV-17, MAV-18, MAV-19, MAV-20, MAV-21, MAV-22, MAV-23, or MAV-24;
b) a junction sequence between an inverted genomic fragment INV and a flanking genomic island;
or homologs thereof;
the probe combined with a label; and
c) analyzing for the presence, if any, of hybridized probe in the test sample, thereby detecting the presence or absence of a mycobacterial strain or phenotype in the test sample.

10. The method of claim 9 wherein the mycobacterial strain is M. paratuberculosis.

11. The method of claim 9 wherein the mycobacterial strain is M. avium.

12. The method of claim 9 wherein the mycobacterial strain causes Johne's disease in animals or Crohn's disease in humans.

13. The method of claim 9 wherein the phenotype is pathogenicity or drug resistance.

14. The method of claim 9 wherein the sample comprises a tissue, collection of cells, cell lysate, body fluid, excretum, in vitro culture, purified polynucleotide, isolated polynucleotide, food sample, medical sample, agro-livestock sample, or environmental sample.

15. The method of claim 9 wherein the target nucleic acid sequence is a junction sequence between an inverted genomic fragment INV-1 and one of the two flanking genomic islands MAV-4 or MAV-19, or homologs thereof.

16. The method of claim 9 wherein the target nucleic acid sequence is a junction sequence between an inverted genomic fragment INV-2 and one of the two flanking genomic islands MAV-21 and MAV-24, or homologs thereof.

17. The method of claim 9 wherein the target nucleic acid sequence is a junction sequence between an inverted genomic fragment INV-3 and one of the two flanking genomic islands MAV-1 and MAV-2, or homologs thereof.
